(19) United States
(12) Patent Application Publication
(10) Pub. No.: US 2007/0048329 A1
(43) Pub. Date: Mar. 1, 2007

(54) Epstein barr virus peptide epitopes, polyepitopes and delivery system therefor

(76) Inventors: Rajiv Khanna, Queensland (AU); Jaikumar Duraiswamy, Queensland (AU)

Correspondence Address: BUCHANAN, INGERSOLL & ROONEY PC, POST OFFICE BOX 1404, ALEXANDRIA, VA, 22313-1404, US

(21) Appl. No.: 10/533,726

(22) PCT Filed: Nov. 3, 2003
(86) PCT No.: PCT/AU03/01451

§ 371 Date: Oct. 25, 2006

(30) Foreign Application Priority Data
Nov. 7, 2002 (AU) 2002952524
Apr. 15, 2003 (AU) 2003901792

(51) Int. Cl. A61K 39/12; C12Q 1/70; C07H 21/04; C12P 21/06; C07K 14/05

(52) U.S. Cl. 424/186.1; 514/44; 435/5; 435/69.1; 435/456; 435/325; 530/350; 536/23.72

(57) ABSTRACT

Immunogenic cytotoxic T cell epitope peptides of Epstein Barr Virus LMP1 protein are provided having between nine and seventeen amino acids and minimal consensus sequences selected from QRH, AGNDG, QNW, VLYS and DSNSNE. These peptides, either individually or in polyepitope constructs, are useful in pharmaceutical compositions, vaccines and methods of treating Epstein Barr Virus associated diseases such as Hodgkin's Disease and/or Nasopharyngeal Carcinoma, although without limitation thereto. A preferred mode of polyepitope delivery is an adenovirus-based system.

### FIELD OF THE INVENTION

THIS INVENTION relates to immunogenic peptides of Epstein Barr Virus. More particularly, this invention relates to cytotoxic T cell epitopes derived from Epstein Barr Virus LMP1 protein. This invention also provides pharmaceutical compositions comprising one or more Epstein Barr Virus cytotoxic T cell epitopes, polyepitopes, an adenovirus-based vaccine delivery system and methods of treating Epstein Barr Virus associated diseases such as Hodgkin's Disease and/or Nasopharyngeal Carcinoma, although without limitation thereto. Also provided is a method of identifying an Epstein Barr Virus cytotoxic T cell epitope.

### BACKGROUND OF THE INVENTION

The Epstein-Barr virus (EBV) is not only one of the most widespread human viruses but, somewhat paradoxically, it is also linked to a range of neoplasms (Anagnostopoulos 1996). These include various B and T cell non-Hodgkin's lymphomas, Hodgkin's disease, and several lymphoepithelioma-like carcinomas, of which nasopharyngeal carcinoma (NPC) is the archetype. The association of EBV with these tumours, and the oncogenic potential of EBV in vitro is well documented (Rickinson 1996; Khanna 2000). CD8⁺ T cell activity has an important role in controlling EBV infections by recognizing small peptides derived from infected cells presented on the surface by MHC class I molecules. EBV specific CTL preparations can be generated in vitro by stimulating memory T cells from peripheral blood of healthy virus carriers with cells of autologous EBV-transformed LCLs (Khanna 1992; Murray 1992). Within an LCL, EBV expresses six nuclear antigens (EBNAs 1, 2, 3A, 3B, 3C, and LP) and two latent membrane proteins (LMP1 and 2). Of these, the EBNA3 family (EBNA 3A, 3B, 3C) is immunodominant for CTL responses over a wide range of HLA backgrounds (Khanna 1992; Murray et al., 1992).

In both HD and NPC, the tumour cells express viral proteins known to provide target epitopes for CTLs. These two malignancies express EBNA1, BARF0, LMP1 and LMP2. EBNA1 includes a unique glycine-alanine repeat (GAr), which acts as a cis-inhibitory signal for proteasomal degradation and thus blocks endogenous presentation of CTL epitopes within this antigen (Levitskaya 1995). CTL presentation of BARF0 epitopes is impaired by the differential splicing of viral transcripts resulting in dominant protein isoforms from which the CTL determinants are deleted (Kienzle 2000). As opposed to EBNA1 and BARF0, both LMP1 and LMP2 are strong targets for EBV-specific CTLs and, therefore, much attention has been directed at identifying target epitopes within these two antigens (Lee 1997; Khanna 1998; Meij 2002).

LMP1 is a membrane spanning protein with cytoplasmic N-terminal and C-terminal domains separated by 6 transmembrane segments. LMP1 has been recognised as one of most crucial latent protein for EBV-mediated transformation of normal B cells and is uniquely able to induce malignant outgrowth and hyperplasia in transgenic mice (Kulwichit 1998). Furthermore, LMP1 is also known to exhibit pleiotropic effects on cellular phenotype of B cells which include induction of activation antigens (Wang 1990), the expression of inhibitors of programmed cell death (Henderson 1991; Laherty 1992), and NF-κB activation through the TRAF signaling pathway (Hammarskjold 1992; Mosialos 1995). Previous studies have shown that LMP1 acts as a constitutively active receptor-like molecule independent of the binding of a ligand. The C-terminus of LMP1 initiates signalling through C-terminal activator regions (referred to as CTAR1, amino acids 194-231 and CTAR2, amino acids 332-386 and CTAR3, amino acids 275-330). CTAR1 & 2 regions are involved in the induction of NF-κB, CTAR2 being the principal NF-κB activator site, while CTAR3 domain was recently reported to bind Janus Kinase 3 (Gires 1999).

### SUMMARY OF THE INVENTION

The present inventors have adopted a novel IFN-γ based assay to conduct an extensive sequence-wide analysis of LMP1-specific T cell responses in a large panel of virus carriers. This approach was combined with functional cytotoxicity analysis to assess the ability of LMP1-specific CTLs to lyse EBV-infected target cells.

In a broad form, the invention provides new and efficacious LMP1-derived EBV peptides potentially suitable for use in immunotherapeutic treatments of neoplasms including B and T cell non-Hodgkin's lymphomas, Hodgkin's disease, and lymphoepithelioma-like carcinomas such as nasopharyngeal carcinoma (NPC).

The invention also provides an adenovirus-based vaccine delivery system that is particularly efficacious when used for the delivery of an EBV polyepitope construct.

The invention therefore provides an isolated EBV CTL peptide epitope comprising at least nine contiguous amino acid residues of an LMP1 protein, wherein said EBV CTL peptide is not YLQQNWWTL (SEQ ID NO:1); ESDSNSNEG (SEQ ID NO:2) or YLLEMLWRL (SEQ ID NO:3).

In a first aspect, the invention provides an isolated EBV CTL peptide epitope comprising the amino acid sequence QRH (SEQ ID NO:4).

In a preferred embodiment, said epitope comprises an amino acid sequence selected from the group consisting of:

| | | |
|---|---|---|
| (i) | QRHSDEHHH; | (SEQ ID NO:9) |
| (ii) | GQRHSDEHH; | (SEQ ID NO:10) |
| (iii) | YYHGQRHSD; | (SEQ ID NO:11) |
| and | | |
| (iv) | WMYYHGQRH. | (SEQ ID NO:12) |

In other embodiments of the first aspect, said epitope comprises an amino acid sequence selected from the group consisting of:

| | | |
|---|---|---|
| (i) | YYHGQRHSDEHH; | (SEQ ID NO:13) |
| (ii) | IWMYYHGQRHSD; | (SEQ ID NO:14) |
| and | | |
| (iii) | LIWMYYHGQRHSDEHHH. | (SEQ ID NO:15) |

In a second aspect, the invention provides an isolated EBV CTL peptide epitope comprising the amino acid sequence AGNDG (SEQ ID NO:5).

In preferred embodiment of the second aspect, said epitope comprises an amino acid sequence selected from the group consisting of:

| | | |
|---|---|---|
| (i) | AGNDGGPPQ; | (SEQ ID NO:16) |
| and | | |
| (ii) | PSDSAGNDG. | (SEQ ID NO:17) |

In other embodiments of the second aspect, said epitope comprises an amino acid sequence selected from the group consisting of:

| | | |
|---|---|---|
| (i) | SDSAGNDGGPPQ; | (SEQ ID NO:18) |
| (ii) | DSAGNDGGPPQL; | (SEQ ID NO:19) |
| and | | |
| (iii) | PHSPSDSAGNDGGPPQL. | (SEQ ID NO:20) |

In a third aspect, the invention provides an isolated EBV CTL peptide epitope comprising the amino acid sequence QNW (SEQ ID NO:6), specifically excluding the sequence YLQQNWWTL (SEQ ID NO:1).

In a preferred embodiment of the third aspect, said epitope comprises and amino acid sequence selected from the group consisting of:

| | | |
|---|---|---|
| (i) | IALYLQQNW; | (SEQ ID NO:21) |
| (ii) | ALYLQQNWW; | (SEQ ID NO:22) |
| (iii) | QNWWTLLVD; | (SEQ ID NO:23) |
| and | | |
| (iv) | LYLQQNWWT. | (SEQ ID NO:24) |

In other embodiments of the third aspect, said epitope comprises an amino acid sequence selected from the group consisting of:

| | | |
|---|---|---|
| (i) | IALYLQQNWWTL; | (SEQ ID NO:25) |
| (ii) | YLQQNWWTLLVD; | (SEQ ID NO:26) |
| and | | |
| (iii) | LIIALYLQQNWWTLLVD. | (SEQ ID NO:27) |

In a fourth aspect, the invention provides an isolated EBV CTL peptide epitope comprising the amino acid sequence VLYS (SEQ ID NO:7).

In a preferred embodiment, said epitope comprises an amino acid sequence selected from the group consisting of:

| | | |
|---|---|---|
| (i) | ALLVLYSFAL; | (SEQ ID NO:28) |
| (ii) | LLVLYSFAL; | (SEQ ID NO:29) |
| (iii) | ALLVLYSFA; | (SEQ ID NO:30) |
| and | | |
| (iv) | VLYSFALML. | (SEQ ID NO:31) |

In other embodiments of the fourth aspect, said epitope comprises an amino acid sequence selected from the group consisting of:

| | | |
|---|---|---|
| (i) | ALLVLYSFALML; | (SEQ ID NO:32) |
| (ii) | GALLVLYSFALM; | (SEQ ID NO:33) |
| (iii) | DWTGGALLVLYS; | (SEQ ID NO:34) |
| (iv) | GGALLVLYSFAL; | (SEQ ID NO:35) |
| and | | |
| (v) | DWTGGALLVLYSFALML. | (SEQ ID NO:36) |

In a fifth aspect, the invention provides an isolated EBV CTL peptide epitope comprising the amino acid sequence DSNSNE (SEQ ID NO:8), specifically excluding the amino acid sequence ESDSNSNEG (SEQ ID NO:2).

In a preferred embodiment of the fifth aspect, the epitope comprises an amino acid sequence selected from the group consisting of:

| | | |
|---|---|---|
| (i) | DSNSNEGRH; | (SEQ ID NO:37) |
| (ii) | SGHESDSNSNEG; | (SEQ ID NO:38) |
| and | | |
| (iii) | TDDSGHESDSNSNEGRH. | (SEQ ID NO:39) |

The invention also provides a variant EBV CTL epitope.

In particular embodiments, said variant has an amino acid sequence set forth in Table 4 (SEQ ID NOS:40-50).

In a sixth aspect, the invention provides an isolated protein comprising at least one EBV CTL epitope according to any of the aforementioned aspects.

Preferably, the isolated protein is a polyepitope protein comprising an amino acid sequence selected from the group consisting of ALLVLYSFA (SEQ ID NO:30) and IALYQQNW (SEQ ID NO:21).

In a preferred embodiment, the isolated polyepitope protein comprises each of the EBV CTL epitopes set forth in Table 5.

In a particularly preferred embodiment, the isolated polyepitope protein comprises the amino acid sequence set forth in SEQ ID NO:81.

In a seventh aspect the invention an isolated nucleic acid encoding the EBV CTL epitope or polyepitope of any of the aforementioned aspects.

In a preferred embodiment, the isolated nucleic acid encodes the polyepitope amino acid sequence set forth in SEQ ID NO:81.

More preferably the isolated nucleic acid of this embodiment has the nucleotide sequence set forth in SEQ ID NO:82.

This aspect also provides an isolated nucleic acid encoding an EBV CTL epitope variant of the aforementioned aspects.

In particular embodiments, said isolated nucleic acids comprise a nucleotide sequence as set forth in Table 4 (SEQ ID NOS: 63-65, 67-69, 71-76 and 78-80).

In an eighth aspect, the invention provides an expression construct comprising the isolated nucleic acid of the sixth aspect operably linked to one or more regulatory nucleotide sequences in an expression vector.

In a preferred embodiment, the expression construct is adenovirus-based.

In a particular embodiment, said expression construct is a polyepitope construct encoding a plurality of EBV CTL epitopes of the invention.

In a ninth aspect, the invention provides a host cell or organism comprising the expression construct of the eighth aspect.

In a tenth aspect, the invention provides a pharmaceutical composition comprising an EBV CTL epitope, or an isolated nucleic acid or expression construct encoding same, according to any of the aforementioned aspects.

Preferably, the pharmaceutical composition is an immunotherapeutic composition.

More preferably, the pharmaceutical composition is a vaccine.

In an eleventh aspect, the invention provides a method of treating an EBV-associated disease, including the step of administering one or more EBV CTL epitopes of the invention, or an expression construct encoding same, to an animal.

The method of this aspect encompasses administration of protein and nucleic acid compositions to therapeutically and/or prophylactically treat EBV associated disease.

Preferred EBV associated diseases include various B and T cell non-Hodgkin's lymphomas, Hodgilin's disease, and several lymphoepithelioma-like carcinomas, of which nasopharyngeal carcinoma (NPC) is a particularly contemplated form, although without limitation thereto.

Preferably, the animal is a mammal.

More preferably, the animal is a human.

In a preferred embodiment, the method of the tenth aspect further includes the step of selecting one or more EBV CTL epitopes according to a HLA type of the human to be treated.

In a twelfth aspect, the invention provides a method of identifying an EBV CTL epitope, said method including the steps of:

(i) producing a plurality of different peptides derived from an LMP1 protein;

(ii) combining said one or more of said peptides with one or more T lymphocytes obtained from an EBV seropositive individual; and

(iii) measuring IFN-y production by said one or more T lymphocytes in response to said one or more peptides, wherein production of IFN-γ above a reference amount is indicative of said one or more peptides having at least one EBV CTL epitope.

In a preferred embodiment, the method of this aspect further includes the step (iv) of determining whether said one or more T lymphocytes produced at step (ii) lyses one or more EBV-infected target cells.

In a thirteenth aspect, the invention provides an antibody which binds one or more EBV CTL epitopes according to the aforementioned aspects of the invention.

In a fourteenth aspect, the invention provides a method of determining whether an animal harbours, or has been exposed to, Epstein Barr Virus, said method including the step of contacting one or more T cells isolated from said individual with one or more EBV peptides of the invention, whereby a response to at least one of the one or more peptides by said one or more T cells indicates that the animal harbours, or has been exposed to, Epstein Barr Virus.

Preferably, the animal is a mammal.

More preferably, the animal is a human.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES AND TABLES

Table 1: BLA antigen (class I) type of the EBV-immune healthy donors or NPC patients included in this specfication.

Table 2: List of LMP1 sequences recognized by EBV-specific T cells from healthy virus carriers.

Table 3: Frequency of T cell responses to MHC class I and class II restricted LMP1 epitopes in ethnically diverse healthy individuals and NPC patients.

Table 4: Sequences of HLA class I-restricted LMP1 epitopes in EBV isolates from Caucasian, African, Chinese, PNG and Indonesian individuals. Encoding nucleotide sequences for each epitope are also shown together with sequence changes present in particular isolates. Peptide sequence variants of SEQ ID NO:30 are designated SEQ ID NO:40. Peptide sequence variants of SEQ ID NO: 21 are designated SEQ ID NOS: 41-43. Peptide sequence variants of YLLEMLWRL (SEQ ID NO: 3) are designated SEQ ID NOS: 44-48. Peptide sequence variants of YLQQNWWTL (SEQ ID NO: 1) are designated SEQ ID NOS: 49 & 50. Nucleotide sequences encoding wild-type and variant epitopes are designated SEQ ID NOS: 62-80.

Table 5: EBV polyepitope peptide sequences and HLA specificity. YLLEMLWRL (SEQ ID NO: 3); YLQQNWWTL (SEQ ID NO: 1); ALLVLYSFA (SEQ ID NO:30); IAYLQQNW (SEQ ID NO:21); SSCSSCPLSKI (SEQ ID NO: 51); PYLFWLAAI (SEQ ID NO:52); TYGPVFMCL (SEQ ID NO:53); RRRWRRLTV (SEQ ID NO:54); LLSAWILTA (SEQ ID NO: 55); LTAGFLIFL (SEQ ID NO:56); VMSNTLLSAW (SEQ ID NO:57); IEDPPFNSL (SEQ ID NO:58); CLGGLLTMV (SEQ ID NO:59).

FIG. 1: Ex vivo profiling of LMP1-specific T cell responses in a panel of healthy seropositive individuals. A: PBMC from healthy seropositive individuals were stimulated with overlapping synthetic peptides (10 μg/ml) from LMP1 and IFN-γ production was measured in ELISPOT assays as described in the Material and Methods section. The results are expressed as spot forming cells (SFC) per 10⁶ PBMC. B: Schematic distribution of T cell reactivity within the LMP1 protein. C: amino acid sequences of the peptide epitopes recognized by healthy virus carriers in ELISPOT assays: peptide 9 (SEQ ID NO: 36); peptide 17 (SEQ ID NO: 60); peptide 21 (SEQ ID NO: 27); peptide 24 (SEQ ID NO: 15); peptide 25 (SEQ ID NO: 61); peptide 27 (SEQ ID NO: 39); peptide 38 (SEQ ID NO:20).

FIG. 2: Ex vivo detection of IFN-γ secretion by CD4⁺ and CD8⁺ T cell populations following stimulation with LMP1 peptides. CD4⁺ cells or CD8⁺ cells were depleted from fresh PBMCs using anti-human CD4 or CD8 immunomagnetic beads as described in the Material and Methods section. These cells were resuspended in growth medium and stimulated with DWTGGALLVLYSFALML (SEQ ID NO: 36; panel A) and TDDSGHESDSNSNEGRH (SEQ ID NO: 39; panel B) peptides (10 μg/ml) and then tested for IFN-γ secretion using ELISPOT assays.

FIG. 3: Mapping of a minimal epitope sequence using ELISPOT assays. PBMC from donors MM (panels A & B), RE (panel C) and LL (panel D) were stimulated with overlapping peptides (5 μg/ml) and IFN-γ production was measured in ELISPOT assays as described in the Material and Methods section. The results are expressed as spot forming cells (SFC) per 10⁶ PBMC.

FIG. 4: Mapping of a minimal epitope sequence using ELISPOT assays. PBMC from donors LL (panel A), MM (panel B), RE (panels C & D) were stimulated with overlapping peptides (1 μg/ml) and IFN-γ production was measured in ELISPOT assays as described in the Material and Methods section. The results are expressed as spot forming cells (SFC) per 10⁶ PBMC.

FIG. 5: A: HLA class I-restriction analysis for IALYLQQNW-specific CTL clone MM22. Autologous and allogeneic EBV transformed LCLs and PHA blasts, some sharing HLA class I alleles with donor MM were exposed to the CTL clone MM22. PHA blasts were presensitized with IALYLQQNW peptide. An effector to target ratio of 2:1 was used in the assay. B: CTL recognition LALYLQQNW epitope by CTL clone MM23. PHA blasts were sensitized with serial dilutions of the peptides and then exposed to IALYLQQNW-specific CTL clone, MM23.

FIG. 6: A: HLA class I-restriction analysis for ALLVLYSFA-specific CTL line from donor LL. Autologous and allogeneic PHA blasts, some sharing HLA class I alleles with donor LL were exposed to the epitope-specific CTL line in the absence or presence of the peptide. An effector to target ratio of 10:1 was used in the assay. B: CTL recognition ALLVLYSFA epitope by CTL line from donor LL. PHA blasts were sensitized with serial dilutions of the peptides and then exposed to ALLVLYSFA-specific CTL line.

FIG. 7: A: CTL recognition of variant and prototypic BLA A2-restricted LMP1 epitope YLLEMLWRL. PHA blasts were sensitized with serial dilutions of each of the peptides and then exposed to either the YLLEMLWRL-specific CTL clone, SB7. B: MHC stabilization analysis on T2 cells using variant and prototypic HLA A2-restricted LMP1 epitope. T2 cells were initially incubated with 200 μl of each of the peptides (10 μg/ml) for 14-16 h at 26° C. followed by incubation at 37° C. for 2-3 h. HLA A2 expression on these cells was analysed by FACS using HLA A2-specific monoclonal antibody.

FIG. 8: Schematic description of the construction of a recombinant adenovirus virus vector that expresses a synthetic DNA encoding (SEQ ID NO: 79) for a polyepitope protein which contains 13 HLA class I-restricted LMP1 and LMP2 epitopes (SEQ ID NO:79; also see Table 5). The DNA sequence encoding this polyepitope protein was constructed using epitope sequence specific primers (referred to as LME-A, LMP-B, LMP-C and LMF-D) and a technique based on mutual priming and overlap extension The nucleic acid sequence of the fragment coded (from the 5′ end) a BamHI restriction site, a Kozak sequence, a methionine start codon, 13 contiguous minimal LMP1 CTL epitopes, a stop codon, and EcoRI restriction site at the 3′ end. The LMP polyepitope insert was excised form the pcDNA3.1 expression vector and cloned into pAdTrack expression vector. After amplification and recombination in E. coli, the Ad5-LMPpoly vector was packaged into infectious adenovirus by transfecting human embryonic kidney (HEK) 293 cells, and recombinant adenovirus harvested by freezing and thawing.

FIG. 9: Endogenous processing of CTL epitopes encoded by Ad5LMP polyepitope.

FIG. 10: Immunogenecity of AdS LMP Polyepitope.

FIG. 11: Immunization with Ad5-LMPpoly affords protection against LMP1 expressing EL4-A2/K^{b} tumour cells. Two groups of six mice were immunized with Ad5-LMPpoly or control adenovirus (10⁸ PFU/mouse) respectively. Twenty-one days post immunization, mice were challenged subcutaneously with EL4-A2/K^{b}-LMP1 cells (10⁷ cell/mouse) and monitored for tumour size for 16 days post challenge. Data is presented as mean±SE of tumour size.

### DETAILED DESCRIPTION OF THE INVENTION

This specification describes a plurality of novel and unexpected EBV CTL epitopes derived from the LMP1 protein. EBV CTL epitopes were identified using a novel IFN-γ based assay combined with functional cytotoxicity analysis to assess the ability of LMP1-specific CTLs to lyse EBV-infected target cells.

Surprisingly, the CTL epitopes of the invention are preferentially derived from the CTAR1 and transmembrane domains of LMP1 protein, to the virtual exclusion of the CTAR2 domain and the N-terminus.

The LMP1-derived EBV peptides of the invention are potentially suitable for use in immunotherapeutic treatments of EBV-associated disease. In particular, the invention contemplates EBV-associated neoplasms including B and T cell non-Hodgkin's lymphomas, Hodgkin's disease, and lymphoepithelioma-like carcinomas such as nasopharyngeal carcinoma (NPC).

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids, or chemically-derivatized amino acids as are well known in the art.

A “peptide” is a protein having no more than fifty (50) amino acids.

A “polypeptide” is a protein having more than fifty (50) amino acids.

By “EBV CTL epitope” is meant a sequence of amino acids that is encoded by an EBV genome and is capable of eliciting an immune response by at least one T cell clonotype when the amino acid sequence is presented to the at least one T cell clonotype in the context of MHC class I in vivo or in vitro. This definition does not exclude T cell epitopes that, in addition, are T helper or B cell epitopes, for example.

The consensus amino acid sequences set forth in SEQ ID NOS:4-9 are minimal, common regions present in groups of EBV CTL peptides of the invention (SEQ ID NOS: 10-39) listed in Table 2.

Suitably, said EBV CTL peptide epitope consists of at least nine (9) and no more than twenty (20) contiguous amino acids.

In certain embodiments, said EBV CTL peptide epitope consists of nine (9), twelve (12) or seventeen (17) contiguous amino acids.

In a preferred embodiment, said EBV CTL peptide epitope has an amino acid sequence selected from the group consisting of: SEQ ID NO:9; SEQ ID NO:10: SEQ ID NO:11; SEQ ID NO:12; SEQ ID NO:16; SEQ ID NO:17; SEQ ID NO:21; SEQ ID NO:22; SEQ ID NO:23; SEQ ID NO:24; SEQ ID NO:28; SEQ ID NO:29; SEQ ID NO:30; SEQ ID NO:31; and SEQ ID NO:37

The invention also contemplates isolated proteins, such as polypeptides or “polyepitope” proteins, comprising one or more, or preferably a plurality of, EBV CTL epitopes of the invention. For example, said epitopes may be present singly or as repeats, which also includes tandemly repeated epitopes. “Spacer” amino acids may also be included between one or more of the EBV CTL epitopes present in said isolated protein.

In one embodiment, an isolated protein may consist of one or more or, preferably, a plurality of, EBV CTL epitopes of the invention.

In another embodiment, an isolated protein may consist essentially of one or more or, preferably, a plurality of, EBV CTL epitopes of the invention.

By “consist essentially of” is meant in this context that the or each EBV CTL peptide epitope has no more than five or preferably no more three amino acids in addition to the EBV CTL epitope sequence.

In the particular context of a polyepitope protein, these additional amino acid residues may be referred to as “spacer” amino acids.

It will also be appreciated that polyepitope proteins of the invention may additionally comprise one or more other EBV CTL peptides, such as one or more of the prior art LMP epitopes described in Table 2 or Table 5 and/or the EBV CTL epitopes YLLEMLWRL (SEQ ID NO:2), YLQQNWWTL (SEQ ID NO:1) and/or ESDSNSNEG (SEQ ID NO:3).

In a preferred embodiment, the isolated polyepitope protein comprises a plurality of MHC Class I-restricted LMP1 and/or LMP2 CTL epitopes.

Preferably, at least one of said CTL epitopes has an amino acid sequence selected from the group consisting of ALLVLYSFA (SEQ ID NO:30) and IALYQQNW (SEQ ID NO:21).

In a particularly preferred embodiment, the isolated polyepitope comprises thirteen EBV CTL epitopes having the respective amino acid sequences YLLEMLWRL (SEQ ID NO: 3); YLQQNWWTL (SEQ ID NO: 1); ALLVLYSFA (SEQ ID NO:30); IAYLQQNW (SEQ ID NO:21); SSCSSCPLSKI (SEQ ID NO: 51); PYLFWLAAI (SEQ ID NO:52); TYGPVFMCL (SEQ ID NO:53); RRRWRRLTV (SEQ ID NO:54); LLSAWILTA (SEQ ID NO: 55); LTAGFLIFL (SEQ ID NO:56); VMSNTLLSAW (SEQ ID NO:57); IEDPPFNSL (SEQ ID NO:58); CLGGLLTMV (SEQ ID NO:59).

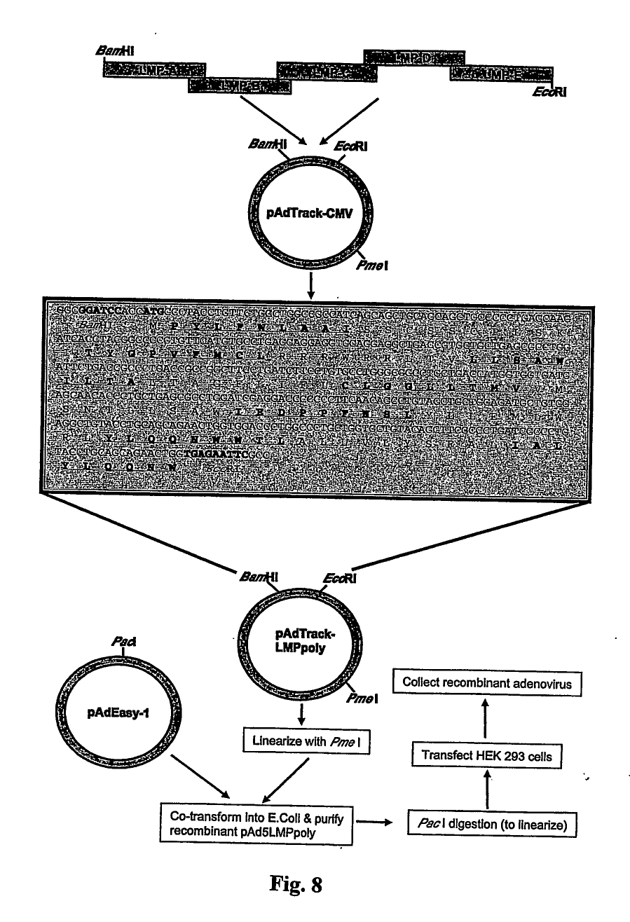

Even more preferably, the isolated polyepitope protein has the amino acid sequence set forth in SEQ ID NO:81 (FIG. 8).

The CTL epitopes in the polyepitope in Table 5 and FIG. 8 have been selected to encompass a broad range of MHC Class I specificities. For example, it is estimated that these optimally-selected HLA specificities would encompass about 90% of the Asian, African and Caucasian populations.

It is also noted that the isolated polyepitope protein in FIG. 8 (SEQ ID NO:81) has been shown by the present inventors to induce CD8+CTL responses in polyepitope-immunized mice, which responses protect mice from tumour challenge.

Also contemplated are EBV CTL epitope variants.

Generally, as used herein, “variants” are EBV CTL epitopes of the invention in which one or more amino acids have been deleted or replaced by different amino acids without substantial alteration to immunogenicity. It is well understood in the art that some amino acids may be changed to others with broadly similar properties without changing the immunogenicity of the peptide (conservative substitutions).

Substantial changes in function are made by selecting substitutions that are less conservative and relatively fewer of these may be tolerated. Generally, the substitutions which are likely to produce the greatest changes in a protein's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser)or no side chain (e.g., Gly).

Specific examples of naturally-occurring variant EBV CTL epitopes are provided in Table 4 and SEQ ID NOS:40-50, which variants are derived from particular ethnoregional EBV isolates and will be discussed in more detail hereinafter.

The invention also contemplates “derivatives” of EBV CTL epitopes of the invention, such as created by chemical modification of amino acid residues, biotinylation, conjugation with fluorochromes, addition of epitope tags (for example c-myc, haemagglutinin and FLAG tags), and fusion partners that facilitate recombinant protein expression, detection and purification (such as glutathione-S-transferase, green fluorescent protein, hexahistidine and maltose-binding protein, although without limitation thereto).

With regard to chemical modification of amino acids, this includes but is not limited to, modification by acylation, amidination, pyridoxylation of lysine, reductive alkylation, trinitrobenzylation of amino groups with 2, 4, 6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto.

In this regard, the skilled person is referred to Chapter 15 of CURRENT PROTOCOLS IN PROTEIN SCIENCE, Eds. Coligan et al. (John Wiley & Sons NY 1995-2000) for more extensive methodology relating to chemical modification of proteins.

EBV CTL epitopes of the invention, EBV polyepitopes and proteins incorporating same may be produced by any means known in the art, including but not limited to, chemical synthesis, recombinant DNA technology and proteolytic cleavage of an LMP protein to produce peptide fragments.

In one embodiment, EBV CTL epitopes may be prepared by chemical synthesis, inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2001). In this regard, reference is also made to International Publication WO 99/02550 and International Publication WO 97/45444.

In another embodiment, recombinant EBV CTL epitopes of the invention, or preferably, proteins comprising EBV CTL epitopes, may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2001), in particular Chapters 10 and 16; and CURRENT PROTOCOLS IN PROTEIN SCIENCE Eds. Coligan et al., (John Wiley & Sons, Inc. NY USA 1995-2001, in particular Chapters 1, 5 and 6.

Nucleic Acids and Expression Constructs

The present invention provides an isolated nucleic acid that encodes an EBV CTL epitope or polyepitope of the invention.

Nucleotide sequences encoding selected EBV CTL epitopes of the invention and variants thereof are set forth in Table 4. Nucleotide sequences encoding other EBV CTL epitopes of the invention may be readily deduced from the complete LMP1-encoding nucleic acid sequence published, for example, under Genbank Accession Number X58140.

The invention also provides nucleotide sequences encoding EBV peptide variants, such as as set forth in Table 4 (SEQ ID NOS: 63-65, 67-69, 71-76 and 78-80).

The nucleotide sequence of an isolated nucleic acid encoding a preferred polyepitope protein is set forth in SEQ ID NO:82 and FIG. 8.

The term “nucleic acid” as used herein designates single-or double-stranded mRNA, RNA, cRNA, RNAi and DNA, DNA inclusive of cDNA and genomic DNA.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides which, for example, is capable of annealing to a complementary nucleic acid “template” and being extended in a template-dependent fashion by the action of a DNA polymerase such as Taq polymerase, RNA-dependent DNA polymerase or Sequenase™.

The present invention also contemplates nucleic acids that have been modified such as by taking advantage of codon sequence redundancy. In a more particular example, codon usage may modified to optimize expression of a nucleic acid in a particular organism or cell type.

The invention also contemplates use of modified purines (for example, inosine, methylinosine and methyladenosine) and modified pyrimines (for example, thiouridine and methylcytosine) in nucleic acids of the invention.

The invention also provides expression constructs that include nucleotide sequences encoding at least one, or preferably a plurality of EBV CTL epitopes of the invention.

Suitably, said expression construct comprises said nucleotide sequences operably linked to one or more regulatory nucleotide sequences in an expression vector.

“Regulatory nucleotide sequences” present in the expression vector may include an enhancer, promoter, splice donor/acceptor signals, Kozak sequence, terminator and polyadenylation sequences, as are well known in the art and facilitate expression of the nucleotide sequence(s) to which they are operably linked, or facilitate expression of an encoded protein.

As used herein, “operably linked” means that said regulatory nucleotide sequence(s) is/are positioned relative to the nucleotide sequence(s) of the invention to initiate, regulate or otherwise control transcription thereof.

Regulatory nucleotide sequences will generally be appropriate for the host cell or organism used for expression. Numerous types of appropriate expression vectors and suitable regulatory sequences are known in the art for a variety of host cells.

With regard to promoters, constitutive promoters (such as CMV, SV40, vaccinia, HTLV1 and human elongation factor promoters) and inducible/repressible promoters (such as tet-repressible promoters and IPTG-, metallothionine- or ecdysone-inducible promoters) are well known in the art and are contemplated by the invention. It will also be appreciated that promoters may be hybrid promoters that combine elements of more than one promoter.

Preferably, said expression construct also includes one or more selectable markers suitable for the purposes of selection of transformed bacteria (such as bla, kanR and tetR) or transformed mammalian cells (such as hygromycin, G418 and puromycin).

The expression construct may also include a fusion partner (typically provided by the expression vector) so that the recombinant protein of the invention is expressed as a fusion protein with said fusion partner. The main advantage of fusion partners is that they assist identification and/or purification of said fusion protein.

Examples of fusion partners have been hereinbefore described. Typically, fusion partners are particularly useful for isolation of a fusion protein by affinity chromatography. For the purposes of fusion polypeptide purification by affinity chromatography, relevant matrices for affinity chromatography are antibody, protein A- or G-, glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

Suitable host cells for expression may be prokaryotic or eukaryotic, such as Escherichia coil (DH5α for example), yeast cells, Sf9 cells utilized with a baculovirus expression system, mammalian cell lines such as lymphoblastoid cell lines, murine EL4 cells and splenocytes isolated from transformed host organisms such as humans and mice, although without limitation thereto.

Expression constructs may be introduced into host cells or organism by any of a number of well known techniques including, but not limited to, transformation by heat shock, electroporation, DEAE-Dextran transfection, microinjection, liposome-mediated transfection, calcium phosphate precipitation, protoplast fusion, microparticle bombardment, viral transformation and the like.

In a particular embodiment, the invention provides an expression construct in the form of a polyepitope expression construct.

Preferably, said polyepitope construct is suitable for use as a DNA vaccine.

According to this embodiment, nucleic acids encoding a plurality of EBV CTL epitopes may, for example, be constructed from synthetic oligonucleotides using a technique such as Splice Overlap by Extension PCR, as described by Thomson et al., 1995, Proc. Natl. Acad. Sci. USA 92 5845.

In particular forms, expression constructs of the invention may be constructs that utilize expression and delivery vectors of viral origin, such as pox viruses and adenoviruses.

When used as a vaccine delivery system, expression constructs of viral origin may be administered to an animal in the form of VLPs or as a “naked” nucleic acid construct.

In one particular embodiment, a polyepitope expression construct according to this embodiment comprises a vaccinia virus promoter, such as the p7.5 promoter present in a plasmid vector. For example, production of a TK-recombinant vaccinia virus using marker rescue recombination is provided in Khanna et al., 1992.

In a more preferred embodiment, the invention provides an adenovirus-based expression construct for use in a vaccine delivery system. Adenovirus-based constructs are capable of infecting a broad spectrum of mammalian and human cells, including both quiescent and proliferating cell types.

Such adenovirus-based expression constructs may comprise a constitutive or inducible/repressible promoter such as by way of a tetracycline inducible/repressible system.

A preferred form of the adenovirus-based expression construct is derived from a replication-incompetent A5 adenovirus lacking at least an E1 gene. An example of such a replication-incompetent adenovirus is provided by the Aden-X system of Clontech.

A particularly preferred adenovirus-based expression construct and vaccine delivery system is provided in detail hereinafter.

Pharmaceutical Compositions and Vaccines

The invention also provides pharmaceutical compositions that comprise one or more EBV CTL epitopes of the invention, inclusive of variants and derivatives thereof, or a nucleic acid expression construct encoding same.

Preferred pharmaceutical compositions are “immunotherapeutic compositions” that provide a therapeutic or prophylactic treatment of EBV-associated diseases as hereinbefore described.

In a particular embodiment, said pharmaceutical composition is a vaccine.

Vaccines may be in the form of proteinacious vaccines comprising one or more EBV CTL epitopes of the invention inclusive of subunit vaccines or in the form of DNA vaccines, a particular example of which is a polyepitope expression construct as hereinbefore described.

Potentially suitable technology relevant to DNA vaccines may be found, for example, in International Publication WO 96/03144 and Thomson et al., 1998, J. Immunol. 160 1717.

Delivery of adenovirus-based constructs may be by systemic administration to an animal or by first transducing antigen-presenting cells, such as dendritic cells, followed by administration of the transduced cells into the animal.

Examples of delivery of adenovirus-transduced dendritic cells for treating EBV-associated disease are provided in Ranieri et al. 1999 and Gahn et al. 2001.

Suitably, the pharmaceutical composition further comprises a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Nack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate, for example, for administration of immunogenic compositions, proteinacious vaccines and DNA vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In the particular case of immunotherapeutic compositions and vaccines, the “pharmaceutically-acceptable carrier, diluent or excipient” may be an adjuvant. As will be understood in the art, an “adjuvant” means a composition comprised of one or more substances that enhances the immunogenicity and efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diptehria toxoid; interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

With regard to subunit vaccines, an example of such a vaccine may be formulated with ISCOMS, such as described in International Publication WO97/45444.

An example of a vaccine in the form of a water-in-oil formulation includes Montanide ISA 720, such as described in International Publication WO97/45444.

Antibodies

The invention also contemplates antibodies against the EBV CTL epitopes of the invention.

Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with a polypeptide, fragment, variant or derivative of the invention. For example, the antibodies may comprise polyclonal antibodies. Such antibodies may be prepared for example by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, spra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies which comprise Fc or Fab fragments of the polyclonal or monoclonal antibodies referred to above. Alternatively, the antibodies may comprise single chain Fv antibodies (scFvs) against the isolated proteins of the invention. Such scFvs may be prepared, for example, in accordance with the methods described respectively in U.S. Pat. No. 5,091,513, European Patent No 239,400 or the article by Winter & Milstein, 1991, Nature 349 293, which are incorporated herein by reference.

Labels may be associated with the antibody or antibody fragment of the invention.

The label may be selected from a group including a chromogen, a catalyst, an enzyme, a fluorophore, a chemiluminescent molecule, a lanthanide ion such as Europium (Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes useful as labels is disclosed in U.S. Patent Specifications U.S. Pat. No. 4,366,241, U.S. Pat. No. 4,843,000, and U.S. Pat. No. 4,849,338, all of which are herein incorporated by reference. Enzyme labels useful in the present invention include alkaline phosphatase, horseradish peroxidase, luciferase, b-galactosidase, glucose oxidase, lysozyme, malate dehydrogenase and the like. The enzyme label may be used alone or in combination with a second enzyme in solution.

By way of example, the fluorophore may be fluorescein isothiocyanate (FITC), oregon green, tetramethylrhodamine isothiocyanate (TRITL), allophycocyanin (APC) and R-Phycoerythrin (RPE), although without limitation thereto.

So that the invention can be readily understood and put into practical effect, the skilled person is directed to the following non-limiting examples wherein Example 1 sets forth the elucidation and immunological characterization of EBV CTL epitopes of the invention and Example 2 sets forth adenovirus-mediated delivery of an EBV polyepitope of the invention.

### EXAMPLES

### Example 1

### Materials and Methods

Establishment and Maintenance of Cell Lines

EBV-transformed LCLs were established from seropositive donors by exogenous virus transformation of peripheral B cells using the B95.8, BL74 and QIM-WIL virus isolates. These cell lines were routinely maintained in RPMI 1640 supplemented with 2 mM L-glutamine, 100 IU/ml penicillin and 100 μg/ml streptomycin plus 10% foetal calf serum (FCS) (growth medium). In addition, the peptide transporter (TAP)-negative B×T hybrid cell line 0.174×CEM.T2 (referred to as T2) (Salter 1986) peptide stabilization assays.

To generate phytohaemagglutinin (PHA) blasts, peripheral blood mononuclear (PBMC) cells were stimulated with PHA (CSL Ltd, Melbourne, Australia) and after 3 days of culture, growth medium containing MLA 144 supernatant and highly purified recombinant human IL-2 (rIL-2) was added (Khanna 1992). PHA blasts were propagated by twice-weekly replacement of rIL-2 and MLA supernatant (no further PHA added) for up to 6 weeks.

Virus Isolates

Lymphoblastoid cell lines (LCLs) were established from a panel of unrelated healthy EBV-seropositive African, Caucasian and PNG donors by spontaneous outgrowth from peripheral lymphocytes cultured in the presence of 0.1 μg/ml cyclosporin A (Moss 1988). A total of 29 spontaneous LCLs (11 Caucasian, 11 PNG, 2 African and 5 South-East Asaian) were used to recover the resident EBV isolate from each individual. In addition, 16 virus isolates from South-East Asia were directly sequenced from EBV carrying nasopharyngeal carcinoma biopsies.

PCR and DNA Sequencing of EBV Gene Fragment

Specific oligonucleotide primers flanking different regions of the LMP1 gene were selected for PCR amplification (Table 1). The resulting PCR products were purified using QIAquick spin columns (Qiagen Inc. Chatsworth, Calif.) and sequenced in both directions using a PRISM ready reaction dyedeoxy terminator cycle sequencing kit (Applied Biosystems Inc., Foster City, Calif.) following the manufacturer's protocol.

Synthetic Peptides

The amino acid sequences of the peptides were derived from the published LMP1 sequences from the Caucasian prototype 1 EBV strain B95.8. A panel of 45 peptides of 17 amino acids in length, overlapping by 8 residues, spanning the entire LMP1 sequence, was synthesised on an automated peptide synthesiser by the Merrifield solid phase method (Mimotopes Pty Ltd, Melbourne, Australia). Peptide aliquots were dissolved in 20% DMSO at 2 mg/ml.

EBV Seropositive Donors

A panel of 42 healthy virus carriers and NPC patients of diverse ethnic origin were recruited for this study. Each of the donors was HLA typed using both serological and PCR-based DNA typing methods (Table 2).

IF-γ-ELISPOT Assay

The ELISPOT assay was used to assess whether stimulation of PBMC from a large panel of seropositive donors with LMP1 peptides could induce IFN-γ expression in T cells (Bharadwaj 2001). Briefly, ninety six-well mixed cellulose ester membrane plates (Millipore, Bedford, USA) were precoated with 100 μL/well of 10 μg/nL of anti-IFN-γ mAb, 1-D1K (Mabtech, Stockhohn, Sweden) overnight at 4° C. The plate was washed six times with phosphate-buffered saline (PBS), and unreactive sites blocked using 5% foetal bovine serum. PBMCs from healthy EBV carriers of known HLA types were separated from whole blood by Ficoll-Hypaque (Sigma) density gradient centrifugation. PBMCs were added in triplicate wells at 2.5×10⁵ cells in a volume of 100 μL/well, and then peptides were added onto the PBMCs at a final 5 μg/mL concentration. PBMCs were also tested at different concentrations in triplicate wells at 2.5×10⁵, 1.5×10⁵ and 1×10⁵ cells/well. PBMCs with various peptide concentrations, 10 μg/mL, 5 μg/mL and 1 μg/mL, were assayed. For negative controls, PBMCs were incubated in growth medium alone without adding peptides. The plate was incubated at 37° C. with 5% CO₂ overnight (about 16-20 hr), then washed thrice with PBST (0.05% of Tween-20 in PBS) and three times with PBS before 100 μL/well of 1 μg/mL anti-IFN-γ mAb, 7-B6-1 (Mabtech, Stockholn, Sweden) was added and incubated at room temperature for 3-4 hr. After incubation, the plate was washed again thrice each with PBST and PBS, and 100 μL/well of 1 μg/mL streptavidin-alkaline phosphatase conjugate (Sigma) was added. The plate was incubated at room temperature for 2 hr. Wells were washed again three times each with PBST and PBS, and individual IFN-γ-producing cells were detected as dark spots after about 30-min to an hour-colour reaction with 5-bromo-4-chloro-3-indolylphosphate and nitroblue tetrazolium using an alkaline phosphatase-conjugated substrate (BCIP/NBT; Sigma). Spots were counted automatically using an image analysis software (ImagePro) (Bharadwaj 2001), and were expressed as spot-forming cells (SFC) per 10⁶ PBMCs. The number of IFN-γ-secreting T cells was calculated by subtracting the negative control value from the SFC count.

Depletion of CD4⁺ and CD8⁺ Cells

CD4⁺ cells or CD8⁺ cells were depleted from fresh PBMCs using anti-human CD4 or CD8 immunomagnetic beads (Dynabeads M450-CD4 and M450-CD8), respectively (Dynal, Oslo, Norway) according to the manufacturer's instructions. Efficient depletion was confirmed by staining the depleted PBMCs using dual staining with FITC-conjugated anti-CD8 and PE-conjugated anti-CD4 antibody and flow cytometry on a Coulter EPICS XL cytometer. The protocol reliably depleted >95% of the CD4⁺ or CD8⁺ cells as confirmed by FACScan analysis. Cells were recounted, resuspended in R10, and set up in triplicate ELISPOT assays.

Establishment of Polyclonal CTL Lines and LMP1-Specific CTL Clones

Polyclonal CTL lines and LMP1-specific CTL clones were established according to previously published methods (Moss 1988; Khanna 1998). Briefly, 2×10⁶ PBMCs were stimulated with 1×10⁶ autologous lymphocytes (responder to stimulator ratio of 2:1) pulsed with 10 μM peptide for 1 hr in 2 ml in a 24-well plate. After 3 days, culture medium with IL-2 (10 U/ml) was added and the cells were further expanded. These lymphocytes were restimulated on day 7 with γ-irradiated (8000 rad) autologous LCLs. After 10 days in culture medium, the cells were used as polyclonal effectors in a standard ⁵¹Cr-release assay against peptide-sensitised autologous PHA blasts.

To generate peripheral blood CTL clones specific for the LMP1 derived peptides, PBMCs of healthy donors were reactivated (2×10⁶) PBMCs with peptide-sensitised (10 ug of peptide/ml) autologous lymphocytes (1×10⁶) in 2 ml wells of a 24-well plate in culture medium. After 3 days, the cells were seeded onto 0.35% agarose and maintained in T cell growth medium containing rIL2 (50 IU/ml). After another 3 days, growing clones were transferred to 96-well round-bottom tissue culture plates (Life Technologies) and cultured in T cell growth medium containing rIL2 (50-100 IU/ml).

T Cell-T Cell Killing

This technique (Burrows, 1992) is based on the ability of cells in CTL culture to present peptide antigens to each other which, given the appropriate peptide, results in CTL-CTL killing. CTL clones were incubated (about 300 cells/well) in 25 μl T cell medium containing 10 μM peptide. Lysis of cells was then assessed after incubation over 3 hr at 37° C. using an inverted microscope.

Cytotoxicity Assays

Target cells were pre-sensitized with synthetic peptide epitopes and then incubated with ⁵¹Cr for 90 min. Following incubation, these cells were washed in growth medium and used as targets in a standard 5 h ⁵¹Cr-release assay (Moss, 1988)

MHC Stabilisation Assays

To assess MHC binding by the HLA A2-restricted LMP1 epitope variants from EBV isolates, T2 cells (2×10⁵) were incubated with 200 μl of each of the peptides (100 μg/ml) at 26° C. for 14-16 h, followed by incubation at 37° C. for 2-3 h. After the incubations, HLA A2 expression was measured by FACS using a specific monoclonal antibody (HB82, ATCC).

### Results

LMP1-Specific T Cell Responses in Ethnically Diverse Healthy Virus Carriers

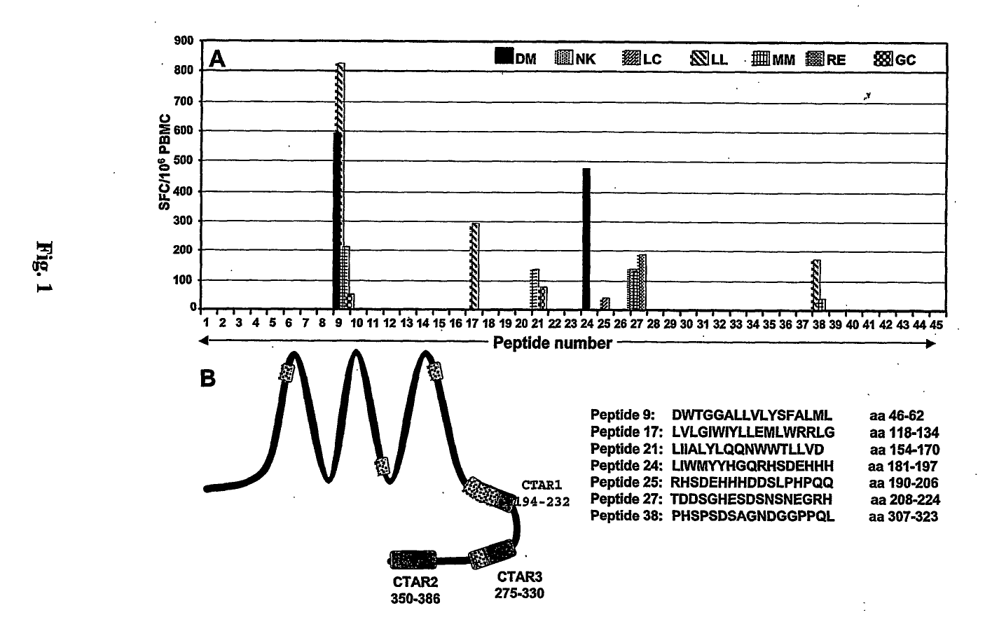

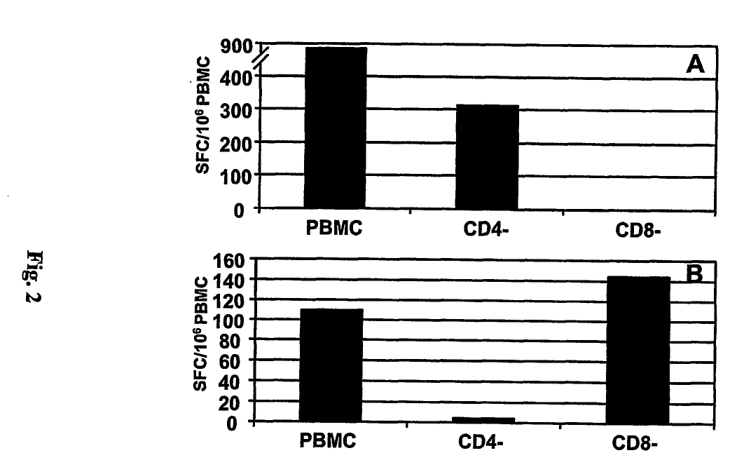

To comprehensively profile the memory T cell responses against the LMP1 in seropositive virus carriers we used the ELISPOT assay, which allowed rapid ex vivo profiling of LMP1-specific immune response without prolonged in vitro culture. PBMC isolated from a panel of seropositive healthy virus carriers (Table 2) were stimulated with the complete set of overlapping LMP1 peptides and the cells that produced IFN-γ were detected. Representative data from the ELISPOT assays using a complete set of overlapping peptides (17 amino acids in length, overlapping by 8 residues) is presented in FIGS. 1A & B. Of the 21 healthy donors assessed, seven donors showed a strong ex vivo response to 7 of the 45 peptides (SFC range 39 to 824/10⁶ PBMCs) (FIG. 1A). One of the interesting features of these responses was that the majority of the T cell reactivity was directed towards the epitopes within the transmembrane and CTAR1 domains of LMP1, while no reactivity was observed towards the CTAR2 and N-terminus (FIG. 1B). In particular, almost 80% of the CTAR1 domain was targeted for T cell recognition. A list of all the 17 mer peptides recognized by the different donors is shown in FIG. 1. To identify the T cell subsets responding to these peptides, PBMCs from the seven donors who recognized 17-mer peptides were fractionated into CD4-depleted and CD8-depleted populations and then tested again in ELISPOT assays. Five of these peptides included reactivity by both CD4+ and CD8+ T cells which suggested that these sequences include both MHC class I- and class II-restricted epitopes. On the other hand peptide DWTGGALLVLYSFALML clearly showed only CD8⁺ T-cell dependent reactivity, while the T cell response to the peptide TDDSGHESDSNSNEGRH was predominantly mediated by CD4+ cells (FIGS. 2A & B). The CD4-dependent reactivity of the peptide TDDSGHESDSNSNEGRH was consistent with the previous observations by Leen and colleagues who mapped an HLA DQ2-restricted epitope within this sequence.

Fine Mapping of LMP1-Specific T Cell Responses

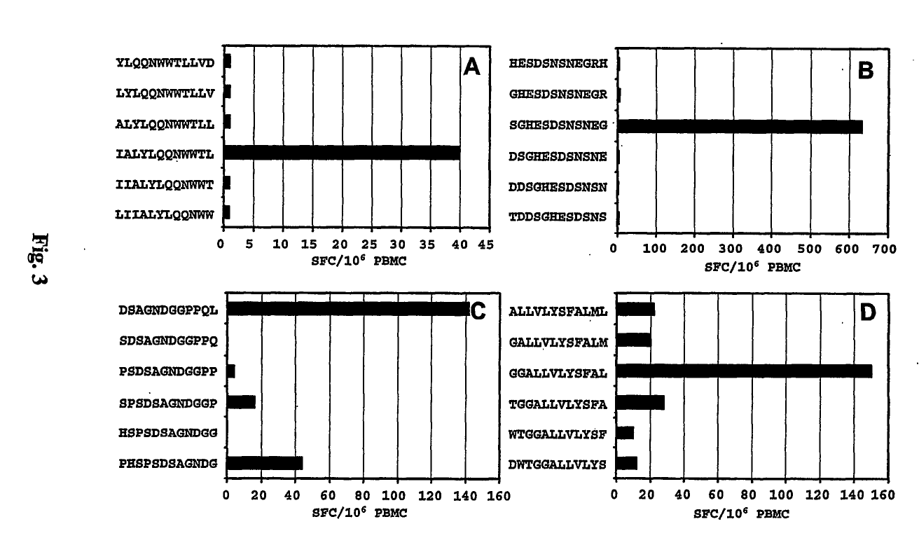

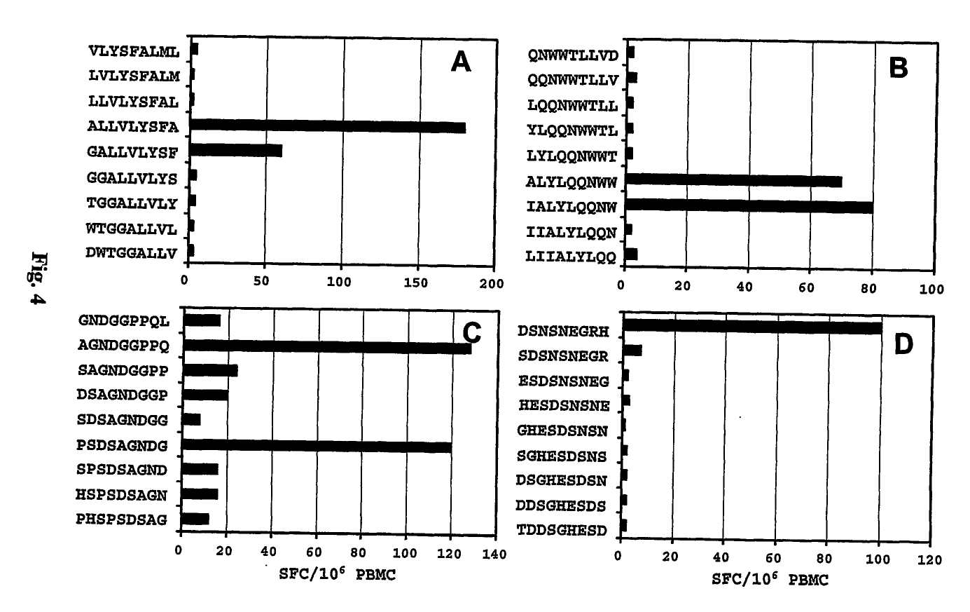

Fine mapping of the LMP1-specific CD8+ T cell responses was achieved using truncations of the 17-mer peptides in ELISPOT assays. In the initial set of experiments, 12-mer overlapping peptides were used to map the immunogenic region within the 17 mer peptides. Representative data from these overlapping peptides for four individual donors are shown in FIGS. 3A-D. A total of twelve 12-mer sequences were identified as potential determinants (see Table 3). Based on these 12-mer peptides, overlapping 9-mer peptides spanning the 12-mer peptides were used to define the minimal epitope sequences. Representative data from four different donors are presented in FIGS. 4A-D. A total of eighteen 9-mer sequences were recognized as potential minimal epitope sequences (Table 3). Two of these minimal sequences (YLLEMLWRL and YLQQNWWTL) have previously been identified as CTL epitopes (Khanna, 1998), while other sequences have been identified for the first time in this study.

Characterization of Novel LMP1 CTL Epitopes

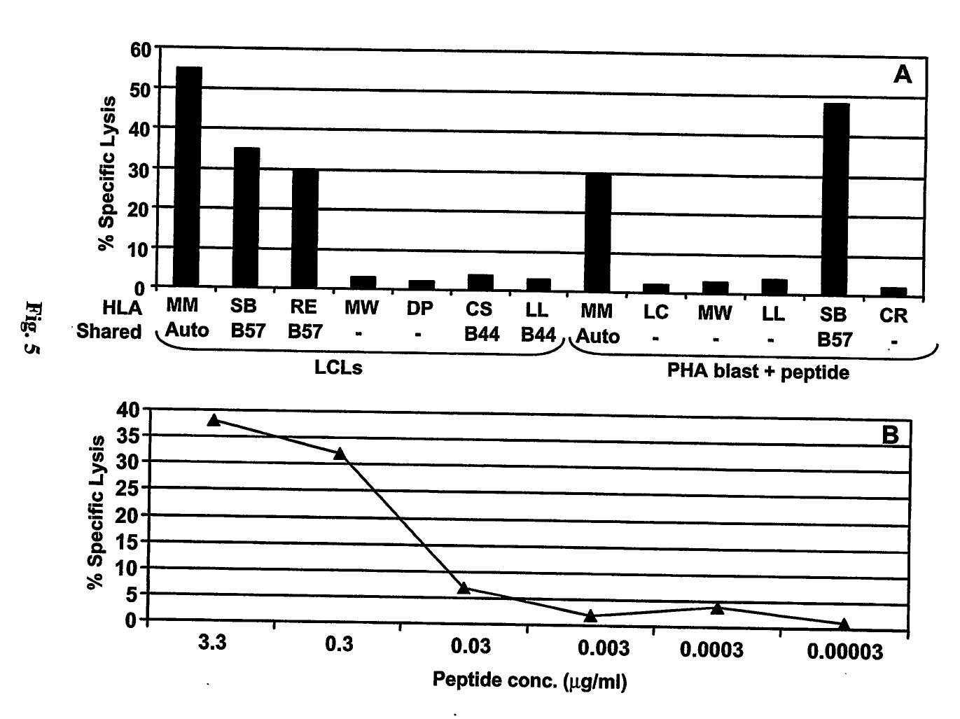

To further characterize the minimal T cell epitopes defined by ELISPOT assays we generated polyclonal and clonal CTL lines specific for these epitopes. PBMCs from the healthy seropositive donors were stimulated with synthetic peptide epitopes and the CTL clones or polyclonal lines were established and tested in standard ⁵¹Cr-release assays. A series of representative data for two of the novel epitopes (IALYLQQNW and ALLVLYSFA) is presented in FIGS. 5 & 6. The IALYLQQNW peptide-specific CTL clones were generated using initial stimulation with peptide-sensitized autologous PBMC followed by continuous restimulation with irradiated autologous LCL. These CTL clones were screened for IALYLQQNW-specific CTL activity by ⁵¹Cr release assays against peptide-sensitised autologous and allogeneic PHA blast and LCLs. Data presented in FIG. 5A clearly showed that target cells sharing only HLA B57 with the CTLs were recognized by the IALYLQQNW-specific CTL clone indicating that this epitope is restricted through the BLA B57 allele. Titration of this minimal epitope on autologous PHA blasts further confirmed the fine specificity of this CTL clone (FIG. 5B).

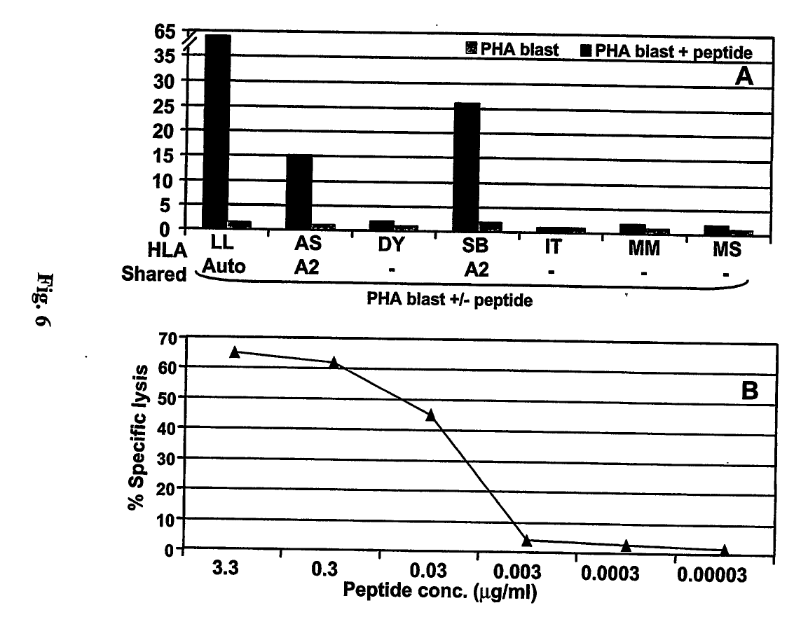

Previous studies with HIV CTL epitopes have shown that similar HLA class I molecules may present the identical peptides as CTL epitopes (Goulder et al., 2000). Since the common subtypes of BLA B57 and BLA B58 differ by only a few amino acids and share similar peptide binding motifs, we explored the possibility that IALTLQQNW may also be restricted through the HLA B58 allele. Indeed, two HLA B58 healthy seropositive donors analysed in our study showed an equally strong response to the minimal IALYLQQNW epitope (Table 3). These observations suggested that this epitope displayed dual HLA restriction for both HLA B57 and HLA B58. Using a similar approach described above, a novel HLA-A2-restricted CTL epitope was identified. The HLA A2 restriction of the ALLVLYSFA epitope was determined using peptide-specific T-cell lines (FIG. 6A). The fine specificity of the ALLVLYSFA epitope-specific CTL clone was subsequently confirmed by dose-response analysis of the minimal peptide (FIG. 6B).

Another 17-mer peptide, TDDSGHESDSNSNEGRH, showed a CD8⁺ T-cell response in Elispot assays in 3/21 healthy donors with the magnitude of responses within the range of 131-190 SFC/10⁶ PBMC (FIG. 1A). Polyclonal CTL lines established from two different donors showed recognition of peptide coated autologous PHA blasts (data not shown). Two donors (MM and RE) recognized minimal peptides, ESDSNSNEG and DSNSNEGRH, respectively, but these minimised epitopes did not generate CTL lines. It is interesting to note that Leen and colleagues have previously mapped an HLA DQ2-restricted T cell epitope within this 17-mer sequence (Table 3).

Recognition of Minimal BLA Class I-Restricted LMP1 CTL Epitopes by Ethnically Diverse Human Subjects

In order to determine the frequency of recognition of the HLA class I-restricted LMP1 CTL epitopes, PBMC from a panel of healthy virus carriers of diverse ethnic origin were screened by ELISPOT assays. A detailed summary of this analysis is presented in Table 3. Four different HLA class I-restricted and one HLA class II-restricted T cell epitopes were included in this analysis. Strong responses were observed against the HLA-A2-restricted YLLEMLWRL, YLQQNWWTL and ALLVLYSFA epitopes in 35-45% of the donors tested. These responses were more consistently seen in Caucasian donors, while these epitopes were less frequently recognized by South-East Asian donors. The HLA-B57-restricted IALYLQQNW epitope was recognized by 5/ HLA B57-positive individuals. Interestingly, some of the HLA B58-positive donors (3/5) also recognized this epitope as efficiently as the HLA B57-positive donors, further confirming the dual HLA class I restriction for this epitope. The HLA class II-restricted LMP1 epitope, TDDSGHESDSNSNEGRH, was recognized by all three healthy virus carriers tested.

Sequence Analysis of HLA Class I-Restricted LMP1 CTL Epitopes in EBV Isolates from Diverse Geographic Regions

Both in the present work and in previous studies from our laboratory (Khanna, 1998), target epitopes in LMP1 were identified using CTLs reactivated with the reference type1 EBV strain, B95.8. However, if such epitopes are to form the basis of an effective CTL therapy for EBV-associated malignancies, we must first determine the extent to which these epitope sequences are conserved among other EBV strains from different world populations. Therefore, a large panel of EBV isolates from healthy virus carriers and NPC biopsies were sequenced across the DNA regions encoding for the LMP1 epitopes. A detailed summary of the sequence analysis of four LMP1 epitopes (IALYLQQNW, ALLVLYSFA, YLLEMLWRL and YLQQNWWTL) is presented in Table 4. The HLA A2-restricted (ALLVLYSFA and YLQQNWWTL) and HLA B57 & B58-restricted (LALYLQQNW) epitopes were highly conserved in virus isolates from Caucasian, African and South-East Asian donors (Table 4). Only a small number of virus isolates showed minor variation within the epitope sequences. Although the YLQQNWWTL and IALYLQQNW epitopes were generally conserved in PNG isolates, alterations in the ALLVLYSFA were more common. Every single isolate showed an identical change from serine to alanine at position 7 (Table 4).

Sequence analysis of the HLA A2-restricted epitope, YLLEMLWRL in EBV isolates from diverse ethnic origins revealed an interesting pattern of genetic variation. Five out of eleven Caucasian isolates showed the prototype B95.8 sequence for the YLLEMLWRL epitope, whereas the remaining isolates displayed changes affecting one or more residues (Table 4). In four isolates, leucine at position 2, methionine at position 5 and arginine at position 8 were substituted for phenylalanine, isoleucine and glycine respectively. The other two isolates showed alterations at positions 2 and 5 or only at position 5. Sequence analysis of virus isolates from South-East Asian individuals revealed an identical set of substitutions in almost all isolates which included alterations at position 2 (L→F), position 5 (M→I). One isolate showed mutation at position 4 (E→D). It is important to stress here that virus isolates from both spontaneous LCLs and NPC biopsies generally displayed identical patterns of mutations (Table 4). No wild-type sequence was detected in any of the isolates from South-East Asia. Interestingly, the dominant genetic variant form of the YLLEMLWRL epitope seen in the South-East Asian population (YFLEILWRL) was also frequently detected in virus isolates from the PNG region.

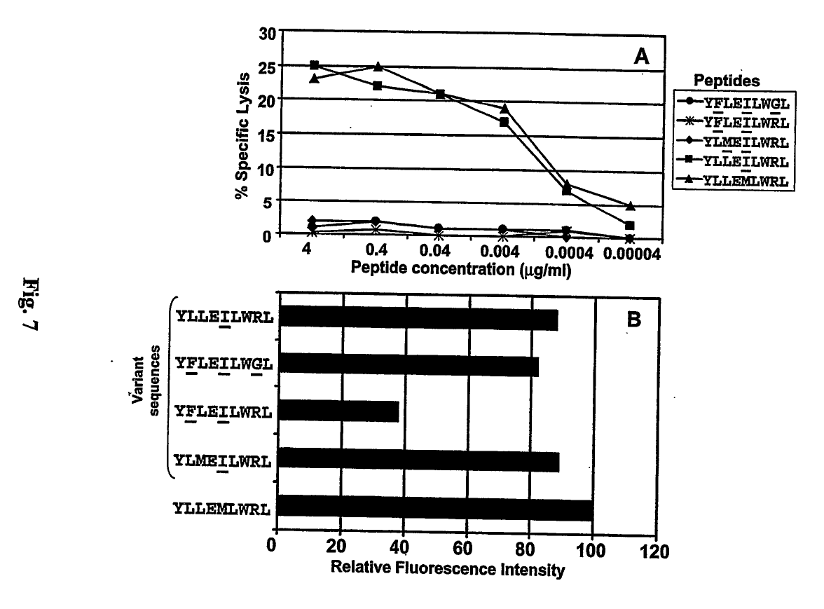

To further characterize the genetic variants of the YLLEMLWRL epitope, synthetic peptides for each of the variant sequences were synthesized and assessed for HLA binding and immunological recognition by EBV-specific CTLs. Data presented in FIG. 7A show that the synthetic peptides for most of the variant sequences were significantly less efficient than the prototypic wild-type YLLEMLWRL peptide at sensitizing autologous PHA blast to lysis by a specific CTL clone. Only one variant sequence, YLLEILWRL was recognized as efficiently as the wild-type epitope (FIG. 7A). Furthermore, incubation of T2 cells with the variant peptide sequence (YFLEILWRL) frequently seen in South-East Asian isolates was unable to rescue HLA A2 expression (FIG. 7B). Other variant peptides (YFLEILWGL, YLMEILWRL and YLLEILWRL), however, significantly increased MHC expression on T2 cells, suggesting that the loss of antigenicity of these variants is due to inappropriate T-cell receptor interaction with the MHC-peptide complex rather than the loss of MHC binding.

### Example 2

### Materials and Methods

Construction of a Recombinant LMP Polyepitope Insert

The DNA sequence coding for the polyepitope amino acid sequence was designed by using universal codons. Five long oligonucleotides (primers 1-5 in the range of 74-100 mer) overlapping by 20 base pairs, representing the polyepitope DNA sequence, were annealed together by using Splicing by Overlap Extension and stepwise asymmetric PCR (see FIG. 8). Briefly, polyepitope sequence specific primers LMPA and LMPB were amplified in a hot started PCR reaction (94° C. for 1 min) for 5 cycles in a 20 μL reaction volume containing elongase enzyme mix and PCR buffers. At the end of 5 cycles, the PCR program was paused at 72° C., 2-μL of reaction was transferred to a new 20 μL reaction already at 72° C. and subjected to a further 5 cycles with primer LMPC and the polyepitope sequence specific forward primer. At cycle 10, the program was paused again; 2 μL of the last reaction was added to new 20 μL reaction and a further 5 cycles was completed with primer LMPD and the polyepitope sequence specific forward primer. This stepwise PCR was repeated until all oligos joined. In the final step, 2 μL of the last reaction was amplified for 25 cycles using polyepitope sequence specific forward and reverse primers.

The nucleic acid sequence of the fragment coded for (from the 5′ end) a Bam HI restriction site, a Kozak consensus sequence, a methionine start codon, 13 contiguous minimal LMP2 and LMP1 CTL epitopes (Table 5), a stop codon, and EcoRI restriction site (FIG. 8). The full-length gel purified PCR fragment was cloned into BamHI/EcoRI site of pcDNA3 expression vector, and checked for mutations by sequencing.

Generation of Ad5-LMP Polyepitope Vaccine Vector

The assembly and production of recombinant Ad5-based adenoviruses was completed using a highly efficient, ligation-based protocol {Mizuguchi 1998 & 1999} of Adeno-X System (CLONTECH, Palo Alto, Calif.) (See FIG. 8). LMP polyepitope insert was ligated into the EcoRI and BamH1 sites of pAd-TrackCMV The recombinant Ad5 vector was then packaged into infectious adenovirus by transfecting human embryonic kidney (HEK) 293 cells, and recombinant adenovirus (referred to as Ad5-LMPpoly) was harvested from transfected cells by freeze-thawing.

Establishment and Maintenance of Cell Lines

EBV-transformed LCLs were established from seropositive donors by exogenous virus transformation of peripheral B cells using the B95.8 virus isolate. These cell lines were routinely maintained in RPMI 1640 (Gibco Invitrogen Corp., Carlsbad, Calif.) supplemented with 2 mM L-glutamine, 100 IU/ml penicillin and 100 μg/ml streptomycin plus 10% FCS (growth medium). In addition, the embryonic kidney cell line 293 {Graham, 1977} was maintained in DMEM containing 10% fetal bovine serum.

Immunization of HLA A2/K^{b} Transgenic Mice with Ad5-LMPPoly Vector

HLA A2/K^{b} transgenic mice used in this study have been described elsewhere (Theobald 1997). These mice express a chimeric class I molecule composed of the alpha 1 & 2 domains of the human A*0201 allele and the alpha 3 domains of the mouse H-2K^{b} class I molecules. These mice were vaccinated intraperitoneally with 10⁸ plaque forming units of recombinant Ad5-LMPpoly or a control adenovirus. After 3 weeks, splenocytes were harvested and tested for epitope-specific T cell responses. These T cell responses were assessed using ELISPOT and in vivo CTL assays as herein described.

Tumour Challenge and Polyepitope Immunization

Two different vaccination strategies were used to assess the efficacy of the LMP polyepitope vaccine. In the first set of experiments, HLA A2/K^{b} mice were immunized intraperitonealy with either Ad5-LMPpoly or control adenovirus (10⁹ PFU/mouse). Three weeks after the immunization, these mice were challenged subcutaneously with live 10⁷ EL4-A2/K^{b}-LMP1 cells. Following challenge, these animals were regularly monitored for 21 days and the tumour size measured by a calliper. In the second set of experimnents, HLA A2/K^{b} mice were first challenged with EL4-A2/K^{b}-LMP1 (10⁷ cells/mouse) tumour cells. Ten days after the challenge, when the tumour size was approximately 0.2 cm in diameter, these mice were immunized with either Ad5-LMPpoly or control adenovirus. The therapeutic efficacy of the LMP polyepitope vaccine was assessed by regular monitoring of tumour regression. Any mice showing tumour size >1.0 cm in diameter were sacrificed according to the guidelines of the institute animal ethics committee.

### Results

Endogenous Processing and CTL Recognition of CTL Epitopes Encoded by Recombinant Adenovirus LMP Polyepitope

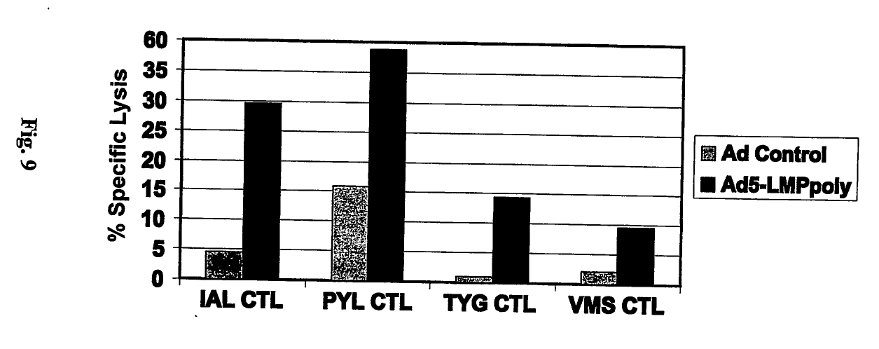

To test whether the LMP epitopes (Table 5) encoded by the polyepitope (Ad5-LMPpoly) were endogenously processed, target cells infected with Ad5-LMPpoly were exposed to LMP1 or LMP2-specific CTL polyclonal lines specific for various epitopes. These CTL lines were generated from healthy virus carriers. HLA matched fibroblast or LCLs infected with Ad5-LMPpoly were efficiently recognized by individual LMP-specific CTL lines (FIG. 9). These results clearly show that HLA class I-restricted CTL epitopes included in the adenovirus LMP polyepitope are efficiently processed and presented to the target cells.

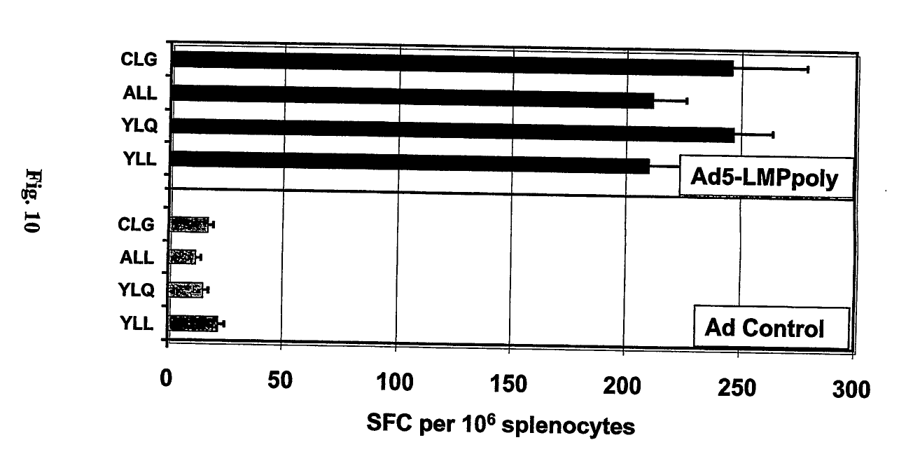

In the next of experiments adenoviral LMP polyepitope was used to restimulate a secondary CTL response in vitro from PBMC obtained from healthy EBV seropositive individuals. The resulting polyclonal cultures were used as effectors against autologous PHA blasts sensitized with LMP1 or LMP2 peptide epitopes. Data presented in FIG. 10 clearly shows that the LMP polyepitope was highly efficient in recalling multiple CTL responses, which were specific for the epitopes restricted by the HLA alleles expressed by each donor. For instance, LMP polyepitope stimulated a strong T cell response to the epitopes YLQ (HLA A2-restricted, LMP1) and CLG (HLA A2-restricted, LMP2), responses were also observed to the epitopes ALL (HLA A2-restricted, LMP1) and YLL (HLA A2-, A68- and A69-restricted, LMP1).

In contrast, stimulation with EBV-transformed LCLs induced very limited expansion of LMP-specific T cells and showed low to undetectable levels of lysis of LMP peptide sensitized target cells (data not shown).

Immunization with Ad5-LMPpoly Vaccine Reverses Outgrowth of LMP1-Expressing Tumours

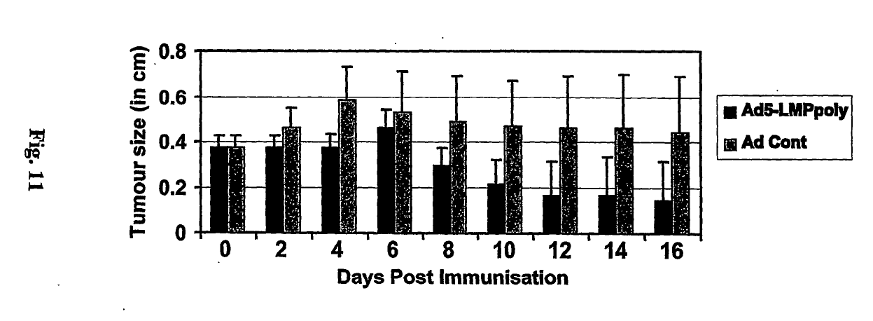

To test whether the adenoviral LMP polyepitope vaccine induced T cell responses can afford protection against LMP1-expressing tumour cells, two groups of HLA A₂/K^{b} mice (10 mice in each group) were first immunized with Ad5-LMPpoly or control adenovirus twice at 14 day interval and then challenged with EL4-A2/-K^{b}-LMP1 cells. These mice were regularly monitored for tumour outgrowth. Although both groups of animals developed tumours, the tumour outgrowth in control adenovirus immunized mice was highly aggressive and showed no evidence of protection from tumour challenge (FIG. 11). It is important to mention here that animals immunized with control adenovirus or Ad5-LMP-poly showed no protection against challenge with EL4/A2K^{b} cells indicating that the epitope-specific immune response was critical for this protection (data not shown).

### Discussion

An important feature common to HD and NPC is the fact that the expression of EBV antigens is often limited to EBNA1, LMP1 and LMP2. Of these, EBNA1 is protected from processing by the classical HLA class I pathway (Levitskaya, 1995) and so LMP1 and LMP2 are the only target antigens available to develop novel strategies to expand antigen-specific T-cell immunity for the treatment of HD and NPC. Therapies aimed at boosting T cell responses to LMP antigens will be useful in the management of EBV-associated relapsed NPC and HD. We adopted the ELISPOT assay to comprehensively profile the LMP1-specific T cell responses in a large panel of virus carriers. Of all the donors tested in this study, LMP1-specific T cell responses were detected in 55-60% of the healthy Caucasians. On the other hand, these responses were less frequently detected in both healthy virus carriers and NPC patients from South East Asia

An interesting feature of the present study was that >80% of T cell reactivity towards LMP1 was directed towards the transmembrane region and CTAR1 domain. Of particular interest was the preferential targeting of CTAR1 domain by multiple donors. Three of seven 15 mer determinants identified by ELISPOT assays were localized within the CTAR1 domain. On the other hand, we were unable to detect any T cell responses to the peptides within the CTAR2 domain located in the distal C-terminus, which is otherwise essential for several LMP1-mediated functions. Previous studies have shown that mutation or deletion of this region alone completely abolishes LMP1-mediated signaling and regulation process. Although the precise reason for this lack of T cell reactivity towards the CTAR2 region is not known, it is possible that EBV might have evolved to protect this functionally critical domain from potential immunological control. An understanding of the mechanism of this evasion could open the possibility of immunologically targeting the CTAR2 domain and thus not only blocking LMP1-mediated transformation but also impairing the latent infection of normal and malignant cells.

Previous studies have suggested that LMP1 sequences in different geographic regions of the world display a very high degree of variation. This genetic variation has been considered as a major impediment towards the use of LMP1 as a potential immunotherapeutic target for the treatment of relapsed HD and NPC. Hence it was important to determine the extent to which the B95.8 derived LMP1 epitope sequences are conserved in EBV isolates from different geographic regions of the world. Studying a large panel of peripheral blood-derived EBV isolates from different regions, as well as NPC biopsies, we found that T cell epitope sequences from LMP1 were generally well conserved. Three of the four epitopes sequenced here showed only minor sequence variation. The only epitope with major sequence divergence was the HLA A2 supertype-restricted YLLEMLWRL epitope, which displayed an interesting pattern of sequence diversity in EBV isolates from different geographic regions. This was particularly highlighted by the isolates from South-East Asia, which showed an identical pattern of genetic variation within the YLLEMLWRL epitope region. Moreover, no difference in the pattern of genetic variation was observed between isolates from NPC and spontaneous LCLs. Although common sequence variants of the YLLEMLWRL epitope were also seen in Caucasian and PNG, these isolates also displayed additional genetic variation which was quite unique to each specific ethnic group. Antigenic analysis of the genetic variants of the YLLEMLWRL epitope indicated that the variant sequence (YFLEILWRL), prevalent in South-East Asia, was not only poorly recognized by epitope-specific CTLs but also showed significant loss in HLA A2 binding when compared to the wild-type sequence. In contrast, although some of the other variant sequences (YFLEILWGL and YLMEILWRL) were poorly recognized, their HLA A2 binding was not significantly affected. Although the precise reason/mechanism for such a high degree of genetic variation within this epitope is not known, it is possible that mutation within this epitope in the isolates from South-East Asia, where NPC is endemic, may provide an advantage in protecting these isolates from the EBV-specific CTL response.

Considering the strong oncogenic potential of LMP1, it is highly unlikely that a vaccine or immunotherapeutic strategy based on full-length LMP1 would ever be a preferred choice for the treatment of NPC and HD. Moreover, the present study demonstrates that in majority of the healthy virus carriers, LMP1 protein generally generates very low levels of T cell responses. This is presumably due to its transmembrane localization which limits its accessibility to the classic MHC class I pathway. The use of highly conserved LMP1 and LMP2 epitopes as a polyepitope vaccine clearly overcomes both these potential limitations.

Accordingly, the present inventors have shown that vaccination with adenovirus vector expressing an LMP polyepitope protein comprising a series of contiguous minimal LMP1 and LMP2 CTL epitopes is capable of inducing multiple independent MHC-restricted CTL responses. These epitopes are not only efficiently processed endogenously by the human cells but also recalled memory CTL response specific for LMP antigens in healthy virus carriers. Furthermore, the adenoviral polyepitope vaccine is also capable of inducing a primary T cell response which was shown to be therapeutic in a tumour challenge system.

It is important to stress here that polyepitope-based vaccine for HD and NPC has number of advantages over the traditionally proposed vaccines, which are based on full-length LMP antigens. Polyepitope proteins are extremely unstable and may be rapidly degraded within the cytoplasm as a result of their limited secondary and tertiary structure. On the other hand the full-length LMP antigens are unlikely to be degraded rapidly and may initiated various intracellular signaling events leading to the development secondary cancers at the site of injection. Another important advantage includes the ability of polyepitope vaccine to induce long-term protective CTL responses against a large number of CTL epitopes using a relatively small construct without any obvious need for a cognate help. Finally, the polyepitope-based vaccine is also likely to overcome any potential problem with the prevalence of LMP1 genetic variants in different ethnic groups of the world.

If a CTL-based therapy for NPC and HD is to be applicable to a significant number of patients, the target population must be presented through HLA alleles present at high frequency in the patient population. In this context, in addition to LMP1, LMP2-specific responses restricted through A11, A24 and B40 are of particular interest because these alleles are very common in the Southern Chinese population (A11, 56%; A24, 27%; B40, 28%), particularly where NPC is endemic. Hence, we have developed a new LMP polyepitope therapeutic vaccine using replication-incompetent adenovirus containing both LMP1 and LMP2 epitopes restricted through common HLA alleles, A2, A11, A23, A24, B27, B40 and B57 present in different ethnic populations. It has been estimated that these optimally selected MHC class I-restricted epitopes would enable more than 90% of the Asian, African and Caucasian populations.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

### REFERENCES

1. Anagnostopoulos, I. and M. Humnmel. 1996. Epstein-Barr virus in tumours. Histopathology 29:297-315.
2. Bharadwaj, M., P. G. Parsons, and D. J. Moss. 2001. Cost-efficient quantification of enzyme-linked immunospot. Biotechniques January 2001.;30.(1.):36.-8. 30:36-38.
3. Burrows, S. R., S. J. Rodda, A. Suhrbier, H. M. Geysen, and D. J. Moss. 1992. The specificity of recognition of a cytotoxic T lymphocyte epitope. Eur. J. Immunol. 22:191-195.
4. Gahn B, Siller-Lopez F, Pirooz A D, Yvon E, Gottschalk S, Longnecker R, Brenner M K, Heslop H E, Aguilar-Cordova E, Rooney C M. 2001. Adenoviral gene transfer into dendritic cells efficiently amplifies the immune response to LMP2A antigen: a potential treatment strategy for Epstein-Barr virus—positive Hodgkin's lymphoma. Int. J. Cancer 93:706-713.
5. Gires, O., F. Kohlluber, E. Kilger, M. Baumann, A. Kieser, C. Kaiser, R. Zeidler, B. Scheffer, M. Ueffing, and W. Hammerschmidt. 1999. Latent membrane protein 1 of Epstein-Barr virus interacts with JAK3 and activates STAT proteins. EMBO J. 18:3064-3073.
6. Goulder, P. J., Y. Tang, S. I. Pelton and B. D. Walker. 2000. HLA-B57-restricted cytotoxic T-lymphocyte activity in a single infected subject toward two optimal epitopes, one of which is entirely contained within the other. J. Virol. 74:5291-5299.
7. Hammarskjöld, M. L. and M. C. Simurda. 1992. Epstein-Barr virus latent membrane protein transactivates the human immunodeficiency virus type 1 long terminal repeat through induction of NF-kappa B activity. J. Virol. 66:6496-6501.
8. Henderson, S., M. Rowe, C. Gregory, D. Croom-Carter, F. Wang, R Longnecker, E. Kieff, and A. B. Rickinson. 1991. Induction of bcl-2 expression by Epstein-Barr virus latent membrane protein 1 protects infected B cells from programmed cell death. Cell 65:1107-1115.
9. Khanna, R., S. R. Burrows, J. Nicholls, L. M. Poulsen. 1998. Identification of cytotoxic T cell epitopes within Epstein-Barr virus (EBV) oncogene latent membrane protein 1 (LMP1): evidence for HLA A2 supertype-restricted immune recognition of EBV-infected cells by LMP1-specific cytotoxic T lymphocytes. Eur. J. Immunol. 28:451-458.
10. Khanna, R. and S. R. Burrows. 2000. Role of cytotoxic t lymphocytes in Epstein-Barr virus-associated diseases. Annu. Rev. Microbiol. 54:19-48.
11. Khanna, R., S. R. Burrows, M. G. Kurilla, C. A. Jacob, I. S. Misko, T. B. Sculley, E. Kieff, and D. J. Moss. 1992. Localization of Epstein-Barr virus cytotoxic T-cell epitopes using recombinant vaccinia: implications for vaccine development. J. Exp. Med. 176:169-176.
12. Kienzle, N., M. Buck, S. L. Silins, S. R. Burrows, D. J. Moss, A. Winterhalter, A. Brooks, and R. Khanna. 2000. Differential splicing of antigen-encoding RNA reduces endogenous epitope presentation that regulates the expansion and cytotoxicity of T cells. J. Immunol. 165:1840-1846.
13. Kulwichit, W., R. H. Edwards, E. M. Davenport, J. F. Baskar, V. Godfrey, and N. Raab-Traub. 1998. Expression of the Epstein-Barr virus latent membrane protein 1 induces B cell lymphoma in transgenic mice. Proc. Natl. Acad. Sci. U.S A. 95:11963-11968.
14. Laherty, C. D., H. M. Hu, A. W. Opipari, F. Wang, and V. M. Dixit. 1992. The Epstein-Barr virus LMP1 gene product induces A20 zinc finger protein expression by activating nuclear factor k B. J. Biol. Chem. 267:24157-24160.
15. Lee, S. P., R. J. Tierney, W. A. Thomas, J. M. Brooks, and A. B. Rickinson. 1997. Conserved CTL epitopes within EBV latent membrane protein 2: a potential target for CTL-based tumor therapy. J. Immunol. 158:3325-3334.
16. Leen, A., P. Meij, I. Redchenko, J. Middeldorp, E. Bloemena, A. Rickinson and N. Blake. 2001. Differential immunogenicity of Epstein-Barr virus latent-cycle proteins for human CD4(+) T-helper 1 responses. J Virol 75:8649-59.
17. Levitskaya, J., M. Coram, V. Levitsky, S. Imreh, P. M. Steigerwald Mullen, G. Klein, M. G. Kurilla, and M. G. Masucci. 1995. Inhibition of antigen processing by the internal repeat region of the Epstein-Barr virus nuclear antigen-1. Nature 375:685-688.
18. Meij, P., A. Leen, A. B. Rickinson, S. Verkoeijen, M. B. Vervoort, E. Bloemena, and J. M. Middeldorp. 2002. Identification and prevalence of CD8(+) T-cell responses directed agalnst Epstein-Barr virus-encoded latent membrane protein 1 and latent membrane protein 2. Int. J. Cancer 99:93-99.
19. Mosialos, G., M. Birkenbach, R Yalamanchili, T. VanArsdale, C. Ware, and E. Kieff. 1995. The Epstein-Barr virus transforming protein LMP1 engages signaling proteins for the tumor necrosis factor receptor family. Cell 80:389-399.
20. Moss, D. J., I. S. Misko, S. R. Burrows, K. Burman, R. McCarthy, and T. B. Sculley. 1988. Cytotoxic T-cell clones discriminate between A- and B-type Epstein-Barr virus transformants. Nature 331:719-721.
21. Murray, N. and A. McMichael. 1992. Antigen presentation in virus infection. Curr. Opin. Immunol. 4:401-407.
22. Ranieri E, Herr W, Gambotto A, Olson W, Rowe D, Robbins P D, Kierstead L S, Watkins S C, Gesualdo L, Storkus W J. 1999. Dendritic cells transduced with an adenovirus vector encoding Epstein-Barr virus latent membrane protein 2B: a new modality for vaccination. J Virol. 73:10416-25.
23. Rickinson, A. B. and E. Kieff. 1996. Epstein-Barr Virus, p. 2397-2446. In B. N. Fields, D. M. Knipe, and P. M. Howley (eds.), Fields Virology. Lippincott-Raven Publishers, Philadelphia.
24. Salter, R. D. and P. Cresswell. 1986. Impaired assembly and transport of HLA-A and -B antigens in a mutant TxB cell hybrid. EMBO J. 5:943-949.
25. Theobald, M. Biggs, J. Hernandez, J. Lustgarten, J. Labadie, C. Sherman L. A. 1997. Tolerance to p53 by A2.1-restricted cytotoxic T lymphocytes. J. Exp. Med. 185:833-841.

26. Wang, F. C. Gregory, C. Sample, M. Rowe, D. Liebowitz, R. Murray, A. Rickinson, E. Kieff. 1990. Epstein-Barr virus latent membrane protein (LMP1) and nuclear proteins 2 and 3C are effectors of phenotypic changes in B lymphocytes: EBNA-2 and LMP1 cooperatively induce CD23. J. Virol. 64:2309-2318.

TABLE 1

| Donor Code | HLA Type | Ethnic Origin |
|---|---|---|
| DM | A24 A29 B44 | Caucasian |
| NK | A2 B14 B44 | Caucasian |
| LC | A1 B8 B18 | Caucasian |
| LL | A2 B7 B44 | Caucasian |
| MM | A1 A3 B44 B57 | Caucasian |
| RE | A1 A3 B50 B57 | Indian |
| GC | A1 A2 B7 B51 | Caucasian |
| MW | A1 A3 B8 B35 | Caucasian |
| DY | A11 A32 B35 B44 | Caucasian |
| TN | A11 A24 B54 B75 | Vietnamese |
| MG | A1 A3 B7 | Caucasian |
| MB | A1 A24 B7 B58 | Indian |
| JD | A31 A33 B35 B58 | Indian |
| IM | A1 A11 B8 B51 | Caucasian |
| LM | A28 A32 B12 | Caucasian |
| SS | A1 A24 B8 B60 | Caucasian |
| SE | A2A29B44B60 | Caucasian |
| SB | A2 B35 B57 | Caucasian |
| CS | A3 A23 B35 B44 | Caucasian |
| JT | A2 A32 B44 B62 | Caucasian |
| MS | A1 A24 B7 B8 | Caucasian |
| DS | A2 B44 B60 | Caucasian |
| WS | A2 A32 B27 B60 | Caucasian |
| EL | A2 A3 B7 B35 | Caucasian |
| KG | A1 A3 B7 B57 | Caucasian |
| JG | Al B8 B57 | Caucasian |
| AS | A2 A3 B7 | Caucasian |
| JF | Not typed | Caucasian |
| CV | Not typed | Vietnamese |
| TL | A2 B13 B58 | Thai/Chinese |
| PR | A2 A24 B35 B60 | Thai/Chinese |
| PA | A2 A11 B60 | Thai |
| WE | A2 A33 B44 B46 | Thai |
| SUr | A2 A3 B13 B61 | Thai |
| CHu | A2 A3 B7 B35 | Thai/Chinese |
| SUm | A2 B57 | Thai |
| SO | A1 A24 B35 B57 | Thai |
| AN | A3 A33 B57 B75 | Thai |
| CHa | A11 A24 B57 B62 | Thai |
| SUc | A11 A24 B51 B58 | Thai/Chinese |
| CI | A2 A33 B46 B58 | |

TABLE 2

| | | | | | | |
|---|---|---|---|---|---|---|
| | | | Minimal | | | |
| Peptide | | 12-mer sequences | sequence | CTL | HLA | |
| Numbr | Epitope Sequence | recognized | recognized | reactivity | Restriction | Reference |

| | | | | | | |
|---|---|---|---|---|---|---|
| 9 | DWTGGALLVLYSFALML | ALLVLYSFALML | ALLVLYSFAL | Y | HLA A2 | |
| | | GALLVLYSFALM | LLVLYSFAL | Y | ND | |
| | | DWTGGALLVLYS | ALLVLYSFA | Y | HLA A2 | |
| | | GGALLVLYSFAL | VLYSFALML | Y | HLA A2 | |
| 17 | LVLGIWIYLLEMLWRRLG | IYLLEMLWRLG | YLLEMLWRL | Y | HLA A2 | Khanna et. al. |
| 21 | LIIALYLQQNWWTLLVD | IALYLQQNWWTL | IALYLQQNW | Y | HLA B571B58 | |
| | | YLQQNWWTLLVD | ALYLQQNWW | Y | ND | |
| | | | YLQQNWWTL | Y | HLA A2 | Khanna et. al. |
| | | | QNWWTLLVD | NT | ND | |
| | | | LYLQQNWWT | NT | ND | |
| 24 | LIWMYYHGQRHSDEHHH | YYHGQRHSDEHH | QRHSDEHHH | ND | ND | |
| | | IWMYYHGQRHSD | GQRHSDEHH | ND | ND | |
| | | | YYHGQRHSD | NT | ND | |
| | | | WMYYHGCQRH | NT | ND | |
| 27 | TDDSGHESDSNSNEGRH | SGHESDSNSNEG | ESDSNSNEG | Y | HLA DQ2 | Leen et. al. |
| | | | DSNSNEGRH | ND | ND | |
| 38 | PHSPSDSAGNDGGPPQL | SDSAGNDGGPPQ | AGNDGGPPQ | ND | ND | |
| | | DSAGNDGGPPQL | PSDSAGNDG | NT | ND | |

TABLE 3

| | HLA type | Subject | Ethnic | CTL response |
|---|---|---|---|---|
| Epitope | of subject | code | Origin | (SFC/10⁶ PBMC) |
| ALLVLYSFA | A2 | LL | Caucasian | 192 |
| | A2 | GC | Caucasian | 96 |
| | A2 | NK | Caucasian | 88 |
| | A2 | SE | Caucasian | 10 |
| | A2 | EL | Caucasian | 80 |
| | A2 | TL | Vietnamese | 128 |
| | A2 | PR | Thai/Chinese | 0 |
| | A2 | PA | Thai/Chinese | 0 |
| | A2 | WE | Thai | 0 |
| | A2 | DS | Caucasian | 1184 |
| | A2 | WS | Caucasian | 592 |
| | A2 | SUr | Thai | 0 |
| | A2 | SB | Caucasian | 480 |
| | A2 | CHu | Thai | 0 |
| | A2 | SUm | Thai/Chinese | 0 |
| | A2 | CI (NPC) | Thai/Chinese | 0 |
| IALYLQQNW | B57 | RE | Indian | 897 |
| | B57 | SB | Caucasian | 455 |
| | B57 | MM | Caucasian | 80 |
| | B57 | SUm | Thai/Chinese | 0 |
| | B57 | KG | Caucasian | 1952 |
| | B57 | SO | Thai | 48 |
| | B57 | AN | Thai | 100 |
| | B57 | Cha | Thai | 0 |
| | B58 | MB | Indian | 240 |
| | B58 | JD | Indian | 955 |
| | B58 | TL | Vietnamese | 504 |
| | B58 | SUc (NPC) | Thai | 0 |
| | B58 | CI (NPC) | Thai/Chinese | 0 |
| TDDSGHESDSNSNEGRH | | | | |
| | HLA | NK | Caucasian | 141 |
| | Class II | MM | Caucasian | 131 |
| | | RE | Indian | 190 |
| YLLEMLWRL | A2 | JF | Caucasian | 0 |
| | A2 | WS | Caucasian | 66 |
| | A2 | CV | Caucasian | 20 |
| | A2 | LL | Caucasian | 42 |
| | A2 | EL | Caucasian | 300 |
| | A2 | PR | Thai/Chinese | 0 |
| | A2 | PA | Thai/Chinese | 0 |
| | A2 | TL | Vietnamese | 136 |
| | A2 | WE | Thai | 0 |
| | A2 | NK | Caucasian | 160 |
| | A2 | AS | Caucasian | 100 |
| | A2 | SUr | Thai | 0 |
| | A2 | CHu | Thai | 0 |
| | A2 | SUm | Thai/Chinese | 0 |
| | A2 | CI (NPC) | Thai/Chinese | 0 |
| YLQQNWWTL | A2 | NK | Caucasian | 244 |
| | A2 | AS | Caucasian | 110 |
| | A2 | LL | Caucasian | 28 |
| | A2 | EL | Caucasian | 100 |
| | A2 | PR | Thai/Chinese | 0 |
| | A2 | PA | Thai/Chinese | 0 |
| | A2 | TL | Vietnamese | 196 |
| | A2 | WE | Thai | 0 |
| | A2 | JF | Caucasian | 44 |
| | A2 | WS | Caucasian | 38 |
| | A2 | CV | Caucasian | 26 |
| | A2 | SUr | Thai | 0 |
| | A2 | CHu | Thai | 0 |
| | A2 | SUm | Thai/Chinese | 0 |
| | A2 | CI (NPC) | Thai/Chinese | 0 |

TABLE 4

| | | HLA | Number of |
|---|---|---|---|
| Virus origin | Epitope sequence | Restriction | Isolates |
| B95.8 | 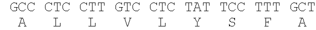 | HLA A2 | |
| Caucasian | 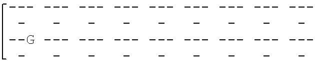 | | 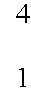 |
| African | 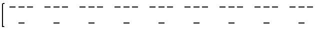 | | 2 |
| S.E. Asian (Spon) | 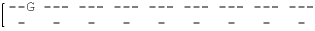 | | 4 |
| S.E. Asian (NPC) | 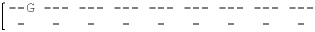 | | 14 |
| PNG | 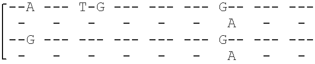 | | 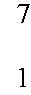 |
| B95.8 | 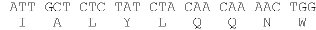 | HLA B57 & B58 | |
| Caucasian | 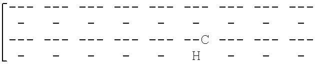 | | 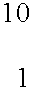 |
| African | 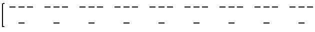 | | 2 |
| S.E. Asian (Spon) | 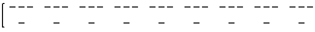 | | 4 |
| S.E. Asian (NPC) | 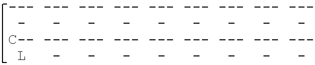 | | 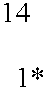 |
| PNG | 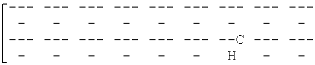 | | 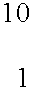 |
| B95.8 | 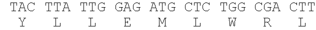 | HLA A2 | |
| Caucasian | 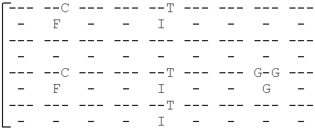 | | 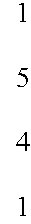 |
| African | 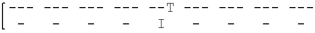 | | 2 |
| S.E. Asian (Spon) | 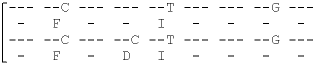 | | 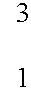 |
| S.E. Asian (NPC) | 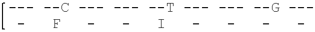 | | 15 |
| PNG | 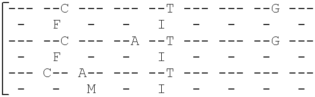 | | 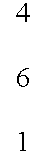 |
| B95.8 | 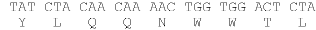 | HLA A2 | |
| Caucasian | 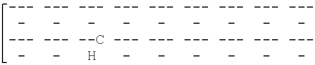 | | 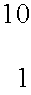 |
| African | 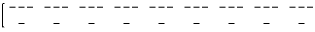 | | 2 |
| S.E. Asian (Spon) | 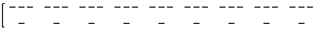 | | 4 |
| S.E. Asian (NPC) | 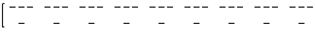 | | 15 |
| PNG | 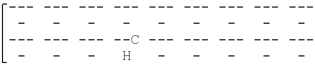 | | 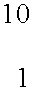 |

TABLE 5

| Epitope | Epitope | EBV | LMP1 | | |
|---|---|---|---|---|---|
| Sequence | code | antigen | Localization | HLA restriction | Reference |
| YLLEMLWRL | YLL | LMP1 | aa125-133 | HLA A2, A68 & | {Khanna1998} |
| | | | | A69 | |
| YLQQNWWTL | YLQ | LMP1 | aa159-167 | HLA A2 | {Khanna1998} |
| ALLVLYSFA | ALL | LMP1 | aa5l-60 | HLA A2 | This study |
| IALYLQQNW | IAL | LMP1 | aal56-164 | HLA B57 & B58 | This study |
| SSCSSCPLSKI | SSC | LMP2 | aa340-350 | HLA A11 | {Lee1997} |
| PYLFWLAAI | PYL | LMP2 | | HLA A23 | {Khanna1996} |
| TYGPVFMCL | TYG | LMP2 | aa419-427 | HLA A24 | {Lee1997} |
| RRRWRRLTV | RRR | LMP2 | aa236-244 | HLA B27 | {Lee1997} |
| LLSAWILTA | LLS | LMP2 | aa447-455 | HLA A2.3 | {Lee2000} |
| LTAGFLIFL | LTA | LMP2 | aa453-461 | HLA 2.1 | {Lee2000} |
| VMSNTLLSAW | VMS | LMP2 | aa442-451 | HLA A25 | {Lee1997} |
| IEDPPFNSL | IED | LMP2 | aa200-208 | HLA B40 | {Lee1997} |
| CLGGLLTMV | CLG | LMP2 | aa426-434 | HLA A2.1 | {Lee1997} |

1. An isolated EBV CTL peptide epitope consisting of up to nine contiguous amino acid residues of an LMP1 protein, and comprising an amino acid sequence selected from the group consisting of:

| | | |
|---|---|---|
| (i) | QRH; | (SEQ ID NO:4) |
| (ii) | AGNDG; | (SEQ ID NO:5) |
| (iii) | QNW, | (SEQ ID NO:6) |
| | specifically excluding the sequence | |
| | YLQQNWWTL; | (SEQ ID NO:1) |
| (iv) | VLYS; | (SEQ ID NO:7) |
| and | | |
| (v) | DSNSNE. | (SEQ ID NO:8) |

2. The isolated EBV CTL peptide epitope of claim 1 consisting of an amino acid sequence selected from the group consisting of:

| | | |
|---|---|---|
| (i) | QRHSDEHHH; | (SEQ ID NO:9) |
| (ii) | GQRHSDEHH; | (SEQ ID NO:10) |
| (iii) | YYHGQRHSD; | (SEQ ID NO:11) |
| and | | |
| (iv) | WMYYHGQRH. | (SEQ ID NO:12) |

3. The isolated EBV CTL epitope of claim 1 consisting of an amino acid sequence selected from the group consisting of:

| | | |
|---|---|---|
| (i) | AGNDGGPPQ; | (SEQ ID NO:16) |
| and | | |
| (ii) | PSDSAGNDG. | (SEQ ID NO:17) |

4. The isolated EBV CTL epitope of claim 1 consisting of an amino acid sequence selected from the group consisting of:

| | | |
|---|---|---|
| (i) | IALYLQQNW; | (SEQ ID NO:21) |
| (ii) | ALYLQQNWW; | (SEQ ID NO:22) |
| (iii) | QNWWTLLVD; | (SEQ ID NO:23) |
| and | | |
| (iv) | LYLQQNWWT. | (SEQ ID NO:24) |

5. The EBV CTL peptide epitope of claim 1 consisting of an amino acid sequence selected from the group consisting of:

| | | |
|---|---|---|
| (i) | LLVLYSFAL; | (SEQ ID NO:29) |
| (ii) | ALLVLYSFA; | (SEQ ID NO:30) |
| and | | |
| (iii) | VLYSFALML. | (SEQ ID NO:31) |

6. The EBV CTL peptide epitope of claim 1 consisting essentially of an amino acid sequence selected from the group consisting of:

| | | |
|---|---|---|
| (i) | DSNSNEGRH; | (SEQ ID NO:37) |
| and | | |
| (ii) | ESDSNSNEG. | (SEQ ID NO:2) |

7. An isolated EBV CTL peptide epitope consisting of an amino acid sequence selected from the group consisting of: ESDSNSNEG (SEQ ID NO:2); QRHSDEHHH (SEQ ID NO:9); GQRHSDEHH (SEQ ID NO:10); YYHGQRHSD (SEQ ID NO:11); WMYYHGQRH (SEQ ID NO:12); YYHGQRHSDEHH (SEQ ID NO:13); IWMYYHGQRHSD (SEQ ID NO:14); LIWMYYHGQRHSDEHHH (SEQ ID NO:15); AGNDGGPPQ (SEQ ID NO:16); PSDSAGNDG (SEQ ID NO:17); SDSAGNDGGPPQ (SEQ ID NO:18); DSAGNDGGPPQL (SEQ ID NO:19); PHSPSDSAGNDGGPPQL (SEQ ID NO:20); IALYLQQNW (SEQ ID NO:21); ALYLQQNWW (SEQ ID NO:22); QNWWTLLVD (SEQ ID NO:23); LYLQQNWWT (SEQ ID NO:24); IALYLQQNWWTL(SEQ ID NO:25); YLQQNWWTLLVD (SEQ ID NO:26); LIIALYLQQNWWTLLVD (SEQ ID NO:27); ALLVLYSFAL(SEQ ID NO:28); LLVLYSFAL (SEQ ID NO:29); ALLVLYSFA (SEQ ID NO:30); VLYSFALML (SEQ ID NO:31); ALLVLYSFALML (SEQ ID NO:32); GALLVLYSFALM (SEQ ID NO:33); DWTGGALLVLYS (SEQ ID NO:34); GGALLVLYSFAL (SEQ ID NO:35); DWTGGALLVLYSFALML (SEQ ID NO:36); DSNSNEGRH (SEQ ID NO:37); SGHESDSNSNEG (SEQ ID NO:38); and TDDSGHESDSNSNEGRH (SEQ ID NO:39).

8. An isolated EBV CTL peptide epitope consisting of an amino acid sequence selected from the group consisting of: SEQ ID NO:2; SEQ ID NO:9; SEQ ID NO:10: SEQ ID NO:11; SEQ ID NO:12; SEQ ID NO:16; SEQ ID NO:17; SEQ ID NO:21; SEQ ID NO:22; SEQ ID NO:23; SEQ ID NO:24; SEQ ID NO:28; SEQ ID NO:29; SEQ ID NO:30; SEQ ID NO:31; and SEQ ID NO:37.

9. A variant of an isolated EBV CTL epitope that consists of an amino acid sequence sequence differing by one, two or three amino acids from an amino acid sequence selected from the group consisting of: SEQ ID NO:1; SEQ ID NO:2; SEQ ID NO:9; SEQ ID NO:10: SEQ ID NO:11; SEQ ID NO:12;SEQ ID NO:16;SEQ ID NO:17;SEQ ID NO:21;SEQ ID NO:22;SEQ ID NO:23; SEQ ID NO:28; SEQ ID NO:29; SEQ ID NO:30; SEQ ID NO:31; and SEQ ID NO:37.

10. The variant of claim 9, comprising an amino acid sequence selected from the group consisting of:
(i) an amino acid sequence differing from SEQ ID NO:1 by one amino acid;
(ii) an amino acid sequence differing from SEQ ID NO:21 by one amino acid; and
(iii) an amino acid sequence differing from SEQ ID NO:30 by one amino acid.

11. A variant of an isolated EBV peptide epitope having an amino acid sequence according to any one of SEQ ID NOS:40-44 and 47-50.

12. An isolated protein comprising at least one EBV CTL epitope and/or a EBV CTL epitope variant according to claim 1.

13. The isolated protein of claim 12, comprising a plurality of EBV CTL epitopes and/or EBV CTL epitope variants.

14. The isolated protein of claim 13 which is a polyepitope protein comprising an amino acid sequence selected from the group consisting of ALLVLYSFA (SEQ ID NO:30) and IALYQQNW (SEQ ID NO:21).

15. The isolated protein of claim 14, wherein a plurality of the EBV CTL epitopes and/or EBV CTL epitope variants are contiguous.

16. The isolated polyepitope protein of claim 15 comprising thirteen EBV CTL epitopes having the respective amino acid sequences YLLEMLWRL (SEQ ID NO: 3); YLQQNWWTL (SEQ ID NO: 1); ALLVLYSFA (SEQ ID NO:30); IAYLQQNW (SEQ ID NO:21); SSCSSCPLSKI (SEQ ID NO: 51); PYLFWLAAI (SEQ ID NO:52); TYGPVFMCL (SEQ ID NO:53); RRRWRRLTV (SEQ ID NO:54); LLSAWILTA (SEQ ID NO: 55); LTAGFLIFL (SEQ ID NO:56); VMSNTLLSAW (SEQ ID NO:57); IEDPPFNSL (SEQ ID NO:58); CLGGLLTMV (SEQ ID NO:59).

17. The isolated polyepitope protein of claim 16 comprising the amino acid sequence set forth in SEQ ID NO:81.

18-26. (canceled)

27. A pharmaceutical composition comprising at least one isolated EBV CTL peptide epitope according to claim 1, together with a pharmaceutically acceptable carrier, diluent or excipient.

28. The pharmaceutical composition of claim 27 comprising an amino acid sequence selected from the group consisting of ALLVLYSFA (SEQ ID NO:30) and IALYQQNW (SEQ ID NO:21).

29. The pharmaceutical composition of claim 27 comprising a polyepitope protein that comprises the amino acid sequence set forth in SEQ ID NO:81.

30-33. (canceled)

34. The pharmaceutical composition of claim 27, which is an immunotherapeutic composition.

35. (canceled)

36. A method of therapeutically and/or prophylactically treating an EBV-associated disease, including the step of administering to an animal at least one isolated EBV CTL epitope according to claim 1.

37. The method of claim 36 wherein the at least one epitope comprises an amino acid sequence selected from the group consisting of ALLVLYSFA (SEQ ID NO:30) and IALYQQNW (SEQ ID NO:21).

38. The method of claim 35 wherein the at least one peptide epitope is a polyepitope protein that comprises the amino acid sequence set forth in SEQ ID NO:81.

39-41. (canceled)

42. The method of claim 36, wherein the EBV associated disease is selected from B and T cell non-Hodgkin's lymphomas, Hodgkin's disease, and lymphoepithelioma-like carcinomas.

43. The method of claim 42, wherein the EBV associated disease is nasopharyngeal carcinoma (NPC).

44. The method of claim 36 wherein the animal is a mammal.

45. The method of claim 44 wherein the mammal is a human.

46. The method of claim 45 wherein one or more of the at least one EBV peptide epitopes is selected according to a HLA type of the human to be treated.

47-54. (canceled)
